(19) United States
(12) Patent Application Publication
(10) Pub. No.: US 2021/0317099 A1
(43) Pub. Date: Oct. 14, 2021

(54) SAFER, POTENT, AND FAST ACTING ANTIMICROBIAL AGENTS

(71) Applicant: Purdue Research Foundation, West Lafayette, IN (US)

(72) Inventors: Padinjaremadhom V. Ramachandran, Lafayette, IN (US); Mohamed Seleem, Blacksburg, VA (US)

(73) Assignee: Purdue Research Foundation, West Lafayette, IN (US)

(21) Appl. No.: 17/220,974

(22) Filed: Apr. 2, 2021

Related U.S. Application Data
(60) Provisional application No. 63/007,545, filed on Apr. 9, 2020.

(51) Int. Cl. C07D 307/33; A61P 31/04; C07D 307/94; C07F 5/02; C07D 405/04; C07D 471/04

(52) U.S. Cl. CPC C07D 307/33; A61P 31/04; C07D 471/04; C07F 5/022; C07D 405/04; C07D 307/94

(57) ABSTRACT

The present invention generally relates to compounds, composition matters, and methods for the treatment of a patient with an infection caused by microorganisms, including bacterial, viral, and fungal infections. In particular, this disclosure relates to safe, highly potent and fast acting antimicrobial agents α-methylene and α-aminomethyl lactones, lactams, iminolactones, and iminolactams, thiolactones, thionolactones, thiolactams, and thionolactams having a formula of I, II, III, or IV, for the treatment of viral and bacterial infections, especially for infections caused by methicillin-resistant Staphylococcus aureus (MRSA).

### CROSS REFERENCE TO RELATED APPLICATIONS

The present U.S. patent application is related to and claims the priority benefit of U.S. Provisional Patent Application Ser. No. 63/007,545, filed Apr. 9, 2020, the contents of which are hereby incorporated by reference in their entirety into the present disclosure.

### TECHNICAL FIELD

The present invention generally relates to compounds, composition matters, and methods for the treatment of a patient with an infection caused by microorganisms, including bacterial, viral, and fungal infections. In particular, this disclosure relates to safe, highly potent and fast acting antimicrobial agents α-methylene and α-aminomethyl lactones, lactams, iminolactones, and iminolactams, thiolactones, thionolactones, thiolactams, and thionolactams for the treatment of viral and bacterial infections, especially for infections caused by methicillin-resistant Staphylococcus aureus (MRSA).

### BACKGROUND

This section introduces aspects that may help facilitate a better understanding of the disclosure. Accordingly, these statements are to be read in this light and are not to be understood as admissions about what is or is not prior art.

Ever since the discovery and application of anti-bacterial to treat infection, bacteria also have evolved and developed resistance to anti-bacterial [1]. A combination of over-use and misuse of antibiotics for humans and in animal feeds has resulted in anti-microbial resistance (AMR) to many classes of antibiotics developed over the past half-century. AMR is a serious health-risk facing every member of the animal kingdom. Methicillin-resistant Staphylococcus aureus (MRSA) has become a leading cause of bacterial infections in both clinical and community settings globally. The increasing occurrence of AMR over the last two decades has resulted in new infection control policies. A recent (2013 [1], updated 2015 [2]) report on “Antibiotic Resistance Threats in the United States”, by the U.S. Department of Health and Human Services Centers for Disease Control and Prevention (CDC) has designated MRSA as a serious bacterial threat [2]. The World Health Organization (WHO) has warned that the pipeline of new drugs is “insufficient to mitigate the threat of antimicrobial resistance” [3]. MRSA is responsible for minor skin infections in healthy people, but it causes serious fatal problems in people lacking immunity [1]. The number and the ratio of death to occurrence is very high for MRSA-related infections, ranking MRSA among the top of all AMR threats. CDC states that if the threat continues at its present rate, then MRSA may change from a serious to an urgent level threat. The structural complexity and/or toxic side effects of the current antibiotics, such as the glycopeptides vancomycin and teicoplanin [4] or the combination drug, quinupristin/dalfopristin [5] thwarts the development of their analogs. Several incidences of resistant strains to these and the oxazolidinone class of antibiotics, e.g. linezolid [4], demand increased efforts for the discovery of novel classes of anti-bacterial agents employing new mode of action. Development of new agents to treat MRSA has become an international priority. Major pharmaceutical companies' decision to shut down antibiotics research projects [6], citing non-profitability, has made the situation extremely risky. There exists an immediate need to develop new classes of antibiotics devoid of any side effects and with enhanced effectiveness to treat MRSA.

### BRIEF DESCRIPTION OF DRAWINGS

FIG. 1: Killing kinetics of compounds 148, 149, 151, 155, and 158 (tested in triplicates at 5×MIC) against methicillin-resistant Staphylococcus aureus (MRSA USA 400) over a 24-hour incubation period at 37° C. DMSO (solvent for the compounds) served as a negative control and vancomycin served as a control drug. The error bars represent standard deviation values obtained from triplicate samples used for each compound/antibiotic studied.

FIG. 2. Analyzing the toxicity of compounds 144, 148, 149, 151, 155 and 158 (tested in triplicates at 16, 32 and 64 μg/mL) against human keratinocytes cells (HaCaT) using the MTS 3-(4,5-dimethylthiazol-2-yl)-5-(3-carboxymethoxyphenyl)-2-(4-sulfophenyl)-2H-tetrazolium) assay. Results are presented as percent viable cells relative to DMSO (negative control to determine a baseline measurement for the cytotoxic impact of each compound). The absorbance values represent an average of three samples analyzed for each compound. Error bars represent standard deviation values. The data were analyzed via a two-way ANOVA with post-hoc Dunnett's test for multiple comparisons. An asterisk (*) denotes statistical significance (P<0.05) between results for compounds and DMSO.

FIG. 3. Analyzing the toxicity of 144, 148, 151, 155, 158, and 187 compounds (tested in triplicates at 16, 32 and 64 μg/mL) against human colorectal cells (Caco-2) using the MTS 3-(4,5-dimethylthiazol-2-yl)-5-(3-carboxymethoxyphenyl)-2-(4-sulfophenyl)-2H-tetrazolium) assay. Results are presented as percent viable cells relative to DMSO (negative control) to determine a baseline measurement for the cytotoxic impact of each compound. The absorbance values represent an average of three samples analyzed for each compound. Error bars represent standard deviation values. The data were analyzed via a two-way ANOVA with post-hoc Dunnett's test for multiple comparisons. An asterisk (*) denotes statistical significance (P<0.05) between results for compounds and DMSO.

FIG. 4. Influence of BHS compounds on the staphylococcal total protease production. OD₆₀₀ was measured following overnight culturing of MRSA USA300 in TSB in presence and absence of 0.25×MIC of 148, 149, 155, 158 and vancomycin followed by incubation of supernatants with skim milk for 1 hour at 37° C. The Data are presented as percent production (means±standard deviation). DMSO (the solvent for the compounds that served as a negative control). Vancomycin served as the control antibiotic. The values represent an average (means±standard deviation) of four samples analyzed for each compound/antibiotic. Error bars represent standard deviation values. An asterisk (*) denotes statistical significance (P<0.05) between results for compounds 148, 149, 155, 158 and vancomycin analyzed via unpaired t test (P<0.5).

FIG. 5. Toxicity analysis of compounds 144, 148, 151, 155 and 187 against murine macrophage (J774) cells using the MTS 3-(4,5-dimethylthiazol-2-yl)-5-(3-carboxymethoxyphenyl)-2-(4-sulfophenyl)-2H-tetrazolium) assay. Results are presented as percent viable mammalian cells (measured as average absorbance ratio relative to DMSO). The absorbance values represent an average of a minimum of three samples analyzed for each compound. Error bars represent standard deviation values for the absorbance values.

FIG. 6. Examination of the activity of compounds 148 on the clearance of intracellular MRSA present inside infected murine macrophage (J774) cells. Data has been presented as login colony forming units of MRSA USA400 per mL inside infected murine macrophages after treatment with 4×MIC and 8×MIC of either compounds 148 or vancomycin (tested in triplicates) for 24 hours. Data were analyzed via two-way ANOVA, with post hoc Dunnet's multiple comparisons test (P<0.05), utilizing GraphPad Prism 6.0 (GraphPad Software, La Jolla, Calif.). Asterisks (*) represent significant difference between treatment of J774 cells with compound 148 in comparison to vancomycin.

### DETAILED DESCRIPTION

While the concepts of the present disclosure are illustrated and described in detail in the description herein, results in the description are to be considered as exemplary and not restrictive in character; it being understood that only the illustrative embodiments are shown and described and that all changes and modifications that come within the spirit of the disclosure are desired to be protected.

The present invention generally relates to compounds useful for the treatment of infectious diseases. Pharmaceutical compositions and methods for treating those diseases are within the scope of this invention.

In some illustrative embodiments, the present invention relates to a method for treating a patient with an infection comprising the step of administering a therapeutically effective amount of one or more compounds of formula (I), or a pharmaceutically acceptable salt thereof, together with one or more carriers, diluents, or excipients, to a patient in need of relief from said infection:

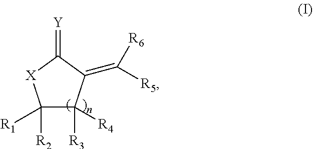

wherein
X═O, NH, NR, S; Y═O, NH, NR, S, wherein R is a C1-C6 alkyl; n=1, 2, 3, 4; and
R₁-R₆ are a substituent independently selected from the group consisting of hydrogen, halogen, hydroxyl, an alkyl, alkenyl, alkynyl, heteroalkyl, heteroalkenyl, heteroalkynyl, heterocyclyl, cycloalkyl, cyclo alkenyl, cycloheteroalkyl, cycloheteroalkenyl, acyl, aryl, heteroaryl, arylalkyl, arylalkenyl, or arylalkynyl, each of which is optionally substituted.

In some illustrative embodiments, the present invention relates to a method for treating a patient with an infection comprising the step of administering a therapeutically effective amount of one or more compounds of formula (II), or a pharmaceutically acceptable salt thereof, together with one or more carriers, diluents, or excipients, to a patient in need of relief from said infection:

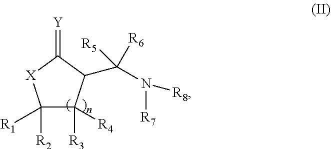

wherein
X═O, NH, NR, S; Y═O, NH, NR, S, wherein R is a C1-C6 alkyl; n=1, 2, 3, 4;
R₁-R₆ are a substituent independently selected from the group consisting of hydrogen, halogen, hydroxyl, an alkyl, alkenyl, alkynyl, heteroalkyl, heteroalkenyl, heteroalkynyl, heterocyclyl, cycloalkyl, cyclo alkenyl, cycloheteroalkyl, cycloheteroalkenyl, acyl, aryl, heteroaryl, arylalkyl, arylalkenyl, or arylalkynyl, each of which is optionally substituted; and
R₇-R₈ are a substituent independently selected from the group consisting of hydrogen, an alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted; or R₇-R₈ are part of a ring system with or without one or more heteroatoms.

In some illustrative embodiments, the present invention relates to a method for treating a patient with an infection comprising the step of administering a therapeutically effective amount of one or more compounds of formula (III), or a pharmaceutically acceptable salt thereof, together with one or more carriers, diluents, or excipients, to a patient in need of relief from said infection:

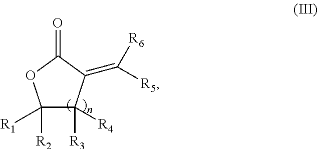

wherein
n=1, 2, 3, 4; and
R₁-R₆ are a substituent independently selected from the group consisting of hydrogen, halogen, hydroxyl, an alkyl, alkenyl, alkynyl, heteroalkyl, heteroalkenyl, heteroalkynyl, heterocyclyl, cycloalkyl, cyclo alkenyl, cycloheteroalkyl, cycloheteroalkenyl, acyl, aryl, heteroaryl, arylalkyl, arylalkenyl, or arylalkynyl, each of which is optionally substituted.

In some illustrative embodiments, the present invention relates to a method for treating a patient with an infection comprising the step of administering a therapeutically effective amount of one or more compounds of formula (IV), or a pharmaceutically acceptable salt thereof, together with one or more carriers, diluents, or excipients, to a patient in need of relief from said infection:

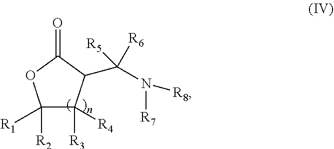

wherein
n=1, 2, 3, 4;
R₁-R₆ are a substituent independently selected from the group consisting of hydrogen, halogen, hydroxyl, an alkyl, alkenyl, alkynyl, heteroalkyl, heteroalkenyl, heteroalkynyl, heterocyclyl, cycloalkyl, cyclo alkenyl, cycloheteroalkyl, cycloheteroalkenyl, acyl, aryl, heteroaryl, arylalkyl, arylalkenyl, or arylalkynyl, each of which is optionally substituted; and
R₇-R₈ are a substituent independently selected from the group consisting of hydrogen, an alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted; or R₇-R₈ are part of a ring system with or without one or more heteroatoms.

In some illustrative embodiments, the present invention relates to a compound for treating a patient with an infection having the formula

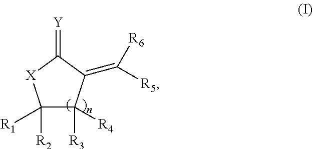

or a pharmaceutically acceptable salt thereof, wherein
X═O, NH, NR, S; Y═O, NH, NR, S, wherein R is a C1-C6 alkyl; n=1, 2, 3, 4; and
R₁-R₆ are a substituent independently selected from the group consisting of hydrogen, halogen, hydroxyl, an alkyl, alkenyl, alkynyl, heteroalkyl, heteroalkenyl, heteroalkynyl, heterocyclyl, cycloalkyl, cyclo alkenyl, cycloheteroalkyl, cycloheteroalkenyl, acyl, aryl, heteroaryl, arylalkyl, arylalkenyl, or arylalkynyl, each of which is optionally substituted.

In some illustrative embodiments, the present invention relates to a compound for treating a patient with an infection having the formula

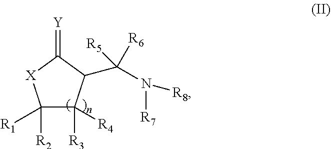

or a pharmaceutically acceptable salt thereof, wherein
X═O, NH, NR, S; Y═O, NH, NR, S, wherein R is a C1-C6 alkyl; n=1, 2, 3, 4;
R₁-R₆ are a substituent independently selected from the group consisting of hydrogen, halogen, hydroxyl, an alkyl, alkenyl, alkynyl, heteroalkyl, heteroalkenyl, heteroalkynyl, heterocyclyl, cycloalkyl, cyclo alkenyl, cycloheteroalkyl, cycloheteroalkenyl, acyl, aryl, heteroaryl, arylalkyl, arylalkenyl, or arylalkynyl, each of which is optionally substituted; and
R₇-R₈ are a substituent independently selected from the group consisting of hydrogen, an alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted; or R₇-R₈ are part of a ring system with or without one or more heteroatoms.

In some illustrative embodiments, the present invention relates to a compound for treating a patient with an infection having the formula

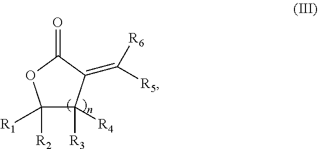

or a pharmaceutically acceptable salt thereof, wherein
n=1, 2, 3, 4; and
R₁-R₆ are a substituent independently selected from the group consisting of hydrogen, halogen, hydroxyl, an alkyl, alkenyl, alkynyl, heteroalkyl, heteroalkenyl, heteroalkynyl, heterocyclyl, cycloalkyl, cyclo alkenyl, cycloheteroalkyl, cycloheteroalkenyl, acyl, aryl, heteroaryl, arylalkyl, arylalkenyl, or arylalkynyl, each of which is optionally substituted.

In some illustrative embodiments, the present invention relates to a compound for treating a patient with an infection having the formula

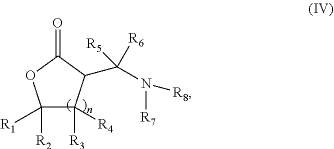

or a pharmaceutically acceptable salt thereof, wherein
n=1, 2, 3, 4;
R₁-R₆ are a substituent independently selected from the group consisting of hydrogen, halogen, hydroxyl, an alkyl, alkenyl, alkynyl, heteroalkyl, heteroalkenyl, heteroalkynyl, heterocyclyl, cycloalkyl, cyclo alkenyl, cycloheteroalkyl, cycloheteroalkenyl, acyl, aryl, heteroaryl, arylalkyl, arylalkenyl, or arylalkynyl, each of which is optionally substituted; and
R₇-R₈ are a substituent independently selected from the group consisting of hydrogen, an alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted; or R₇-R₈ are part of a ring system with or without one or more heteroatoms.

In some illustrative embodiments, the present invention relates to a pharmaceutical composition comprising one or more compounds disclosed herein, together with one or more pharmaceutically acceptable diluents, excipients or carriers.

In some illustrative embodiments, the present invention relates to a pharmaceutical composition comprising nanoparticles of one or more compounds of disclosed herein, together with one or more diluents, excipients or carriers.

In some other embodiments, the present invention relates to a method of use of a compound or a pharmaceutically acceptable salt thereof disclosed herein in the manufacture of a medicament for treating infection in a subject.

As used herein, the following terms and phrases shall have the meanings set forth below. Unless defined otherwise, all technical and scientific terms used herein have the same meaning as commonly understood to one of ordinary skill in the art.

In the present disclosure the term “about” can allow for a degree of variability in a value or range, for example, within 10%, within 5%, or within 1% of a stated value or of a stated limit of a range. In the present disclosure the term “substantially” can allow for a degree of variability in a value or range, for example, within 90%, within 95%, 99%, 99.5%, 99.9%, 99.99%, or at least about 99.999% or more of a stated value or of a stated limit of a range.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading may occur within or outside of that particular section. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

The term “substituted” as used herein refers to a functional group in which one or more hydrogen atoms contained therein are replaced by one or more non-hydrogen atoms. The term “functional group” or “substituent” as used herein refers to a group that can be or is substituted onto a molecule. Examples of substituents or functional groups include, but are not limited to, a halogen (e.g., F, Cl, Br, and I); an oxygen atom in groups such as hydroxyl groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, azides, hydroxylamines, cyano, nitro groups, N-oxides, hydrazides, and enamines; and other heteroatoms in various other groups.

The term “alkyl” as used herein refers to substituted or unsubstituted straight chain and branched alkyl groups and cycloalkyl groups having from 1 to about 20 carbon atoms (C₁-C₂₀), 1 to 12 carbons (C₁-C₁₂), 1 to 8 carbon atoms (C₁-C₈), or, in some embodiments, from 1 to 6 carbon atoms (C₁-C₆). Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl and isoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkenyl” as used herein refers to substituted or unsubstituted straight chain and branched divalent alkenyl and cycloalkenyl groups having from 2 to 20 carbon atoms (C₂-C₂₀), 2 to 12 carbons (C₂-C₁₂), 2 to 8 carbon atoms (C₂-C₈) or, in some embodiments, from 2 to 4 carbon atoms (C₂-C₄) and at least one carbon-carbon double bond. Examples of straight chain alkenyl groups include those with from 2 to 8 carbon atoms such as —CH═CH—, —CH═CHCH₂—, and the like. Examples of branched alkenyl groups include, but are not limited to, —CH═C(CH₃)— and the like.

An alkynyl group is the fragment, containing an open point of attachment on a carbon atom that would form if a hydrogen atom bonded to a triply bonded carbon is removed from the molecule of an alkyne. The term “hydroxyalkyl” as used herein refers to alkyl groups as defined herein substituted with at least one hydroxyl (—OH) group.

The term “cycloalkyl” as used herein refers to substituted or unsubstituted cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. In some embodiments, cycloalkyl groups can have 3 to 6 carbon atoms (C₃-C₆). Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like.

The term “acyl” as used herein refers to a group containing a carbonyl moiety wherein the group is bonded via the carbonyl carbon atom. The carbonyl carbon atom is also bonded to another carbon atom, which can be part of a substituted or unsubstituted alkyl, aryl, aralkyl cycloalkyl, cycloalkylalkyl, heterocyclyl, heterocyclylalkyl, heteroaryl, heteroarylalkyl group or the like. In the special case wherein the carbonyl carbon atom is bonded to a hydrogen, the group is a “formyl” group, an acyl group as the term is defined herein. An acyl group can include 0 to about 12-40, 6-10, 1-5 or 2-5 additional carbon atoms bonded to the carbonyl group. An acryloyl group is an example of an acyl group. An acyl group can also include heteroatoms within the meaning here. A nicotinoyl group (pyridyl-3-carbonyl) is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “aryl” as used herein refers to substituted or unsubstituted cyclic aromatic hydrocarbons that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons (C₆-C₁₄) or from 6 to 10 carbon atoms (C₆-C₁₀) in the ring portions of the groups. Aryl groups can be unsubstituted or substituted, as defined herein. Representative substituted aryl groups can be mono-substituted or substituted more than once, such as, but not limited to, 2-, 3-, 4-, 5-, or 6-substituted phenyl or 2-8 substituted naphthyl groups, which can be substituted with carbon or non-carbon groups such as those listed herein.

The term “aralkyl” and “arylalkyl” as used herein refers to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The term “heterocyclyl” as used herein refers to substituted or unsubstituted aromatic and non-aromatic ring compounds containing 3 or more ring members, of which, one or more is a heteroatom such as, but not limited to, B, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. In some embodiments, heterocyclyl groups include heterocyclyl groups that include 3 to 8 carbon atoms (C₃-C₈), 3 to 6 carbon atoms (C₃-C₆) or 6 to 8 carbon atoms (C₆-C₈).

A heteroaryl ring is an embodiment of a heterocyclyl group. The phrase “heterocyclyl group” includes fused ring species including those that include fused aromatic and non-aromatic groups. Representative heterocyclyl groups include, but are not limited to pyrrolidinyl, azetidinyl, piperidynyl, piperazinyl, morpholinyl, chromanyl, indolinonyl, isoindolinonyl, furanyl, pyrrolidinyl, pyridinyl, pyrazinyl, pyrimidinyl, triazinyl, thiophenyl, tetrahydrofuranyl, pyrrolyl, oxazolyl, oxadiazolyl, imidazolyl, triazyolyl, tetrazolyl, benzoxazolinyl, benzthiazolinyl, and benzimidazolinyl groups.

The term “heterocyclylalkyl” as used herein refers to alkyl groups as defined herein in which a hydrogen or carbon bond of an alkyl group as defined herein is replaced with a bond to a heterocyclyl group as defined herein. Representative heterocyclylalkyl groups include, but are not limited to, furan-2-yl methyl, furan-3-yl methyl, pyridine-3-yl methyl, tetrahydrofuran-2-yl methyl, and indol-2-yl propyl.

The term “heteroarylalkyl” as used herein refers to alkyl groups as defined herein in which a hydrogen or carbon bond of an alkyl group is replaced with a bond to a heteroaryl group as defined herein.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can further include double or triple bonds, and can also include heteroatoms. For example, an allyloxy group is an alkoxy group within the meaning herein. A methoxyethoxy group is also an alkoxy group within the meaning herein, as is a methylenedioxy group in a context where two adjacent atoms of a structure are substituted therewith.

The term “amine” as used herein refers to primary, secondary, and tertiary amines having, e.g., the formula N(group)₃ wherein each group can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

The term “amino group” as used herein refers to a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The terms “halo,” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, —CF(CH₃)₂ and the like.

The term “optionally substituted,” or “optional substituents,” as used herein, means that the groups in question are either unsubstituted or substituted with one or more of the substituents specified. When the groups in question are substituted with more than one substituent, the substituents may be the same or different. When using the terms “independently,” “independently are,” and “independently selected from” mean that the groups in question may be the same or different. Certain of the herein defined terms may occur more than once in the structure, and upon such occurrence each term shall be defined independently of the other.

The compounds described herein may contain one or more chiral centers, or may otherwise be capable of existing as multiple stereoisomers. It is to be understood that in one embodiment, the invention described herein is not limited to any particular stereochemical requirement, and that the compounds, and compositions, methods, uses, and medicaments that include them may be optically pure, or may be any of a variety of stereoisomeric mixtures, including racemic and other mixtures of enantiomers, other mixtures of diastereomers, and the like. It is also to be understood that such mixtures of stereoisomers may include a single stereochemical configuration at one or more chiral centers, while including mixtures of stereochemical configuration at one or more other chiral centers.

Similarly, the compounds described herein may include geometric centers, such as cis, trans, E, and Z double bonds. It is to be understood that in another embodiment, the invention described herein is not limited to any particular geometric isomer requirement, and that the compounds, and compositions, methods, uses, and medicaments that include them may be pure, or may be any of a variety of geometric isomer mixtures. It is also to be understood that such mixtures of geometric isomers may include a single configuration at one or more double bonds, while including mixtures of geometry at one or more other double bonds.

As used herein, the term “salts” and “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic groups such as amines; and alkali or organic salts of acidic groups such as carboxylic acids. Pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic, and the like.

Pharmaceutically acceptable salts can be synthesized from the parent compound which contains a basic or acidic moiety by conventional chemical methods. In some instances, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, the disclosure of which is hereby incorporated by reference.

The term “solvate” means a compound, or a salt thereof, that further includes a stoichiometric or non-stoichiometric amount of solvent bound by non-covalent intermolecular forces. Where the solvent is water, the solvate is a hydrate.

The term “prodrug” means a derivative of a compound that can hydrolyze, oxidize, or otherwise react under biological conditions (in vitro or in vivo) to provide an active compound, particularly a compound of the invention. Examples of prodrugs include, but are not limited to, derivatives and metabolites of a compound of the invention that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Specific prodrugs of compounds with carboxyl functional groups are the lower alkyl esters of the carboxylic acid. The carboxylate esters are conveniently formed by esterifying any of the carboxylic acid moieties present on the molecule. Prodrugs can typically be prepared using well-known methods, such as those described by Burger's Medicinal Chemistry and Drug Discovery 6th ed. (Donald J. Abraham ed., 2001, Wiley) and Design and Application of Prodrugs (H. Bundgaard ed., 1985, Harwood Academic Publishers GmbH).

Further, in each of the foregoing and following embodiments, it is to be understood that the formulae include and represent not only all pharmaceutically acceptable salts of the compounds, but also include any and all hydrates and/or solvates of the compound formulae or salts thereof. It is to be appreciated that certain functional groups, such as the hydroxy, amino, and like groups form complexes and/or coordination compounds with water and/or various solvents, in the various physical forms of the compounds. Accordingly, the above formulae are to be understood to include and represent those various hydrates and/or solvates. In each of the foregoing and following embodiments, it is also to be understood that the formulae include and represent each possible isomer, such as stereoisomers and geometric isomers, both individually and in any and all possible mixtures. In each of the foregoing and following embodiments, it is also to be understood that the formulae include and represent any and all crystalline forms, partially crystalline forms, and non-crystalline and/or amorphous forms of the compounds.

The term “pharmaceutically acceptable carrier” is art-recognized and refers to a pharmaceutically-acceptable material, composition or vehicle, such as a liquid or solid filler, diluent, excipient, solvent or encapsulating material, involved in carrying or transporting any subject composition or component thereof. Each carrier must be “acceptable” in the sense of being compatible with the subject composition and its components and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

As used herein, the term “administering” includes all means of introducing the compounds and compositions described herein to the patient, including, but are not limited to, oral (po), intravenous (iv), intramuscular (im), subcutaneous (sc), transdermal, inhalation, buccal, ocular, sublingual, vaginal, rectal, and the like. The compounds and compositions described herein may be administered in unit dosage forms and/or formulations containing conventional nontoxic pharmaceutically acceptable carriers, adjuvants, and vehicles.

Illustrative formats for oral administration include tablets, capsules, elixirs, syrups, and the like. Illustrative routes for parenteral administration include intravenous, intraarterial, intraperitoneal, epidural, intraurethral, intrasternal, intramuscular and subcutaneous, as well as any other art recognized route of parenteral administration.

Illustrative means of parenteral administration include needle (including microneedle) injectors, needle-free injectors and infusion techniques, as well as any other means of parenteral administration recognized in the art. Parenteral formulations are typically aqueous solutions which may contain excipients such as salts, carbohydrates and buffering agents (preferably at a pH in the range from about 3 to about 9), but, for some applications, they may be more suitably formulated as a sterile non-aqueous solution or as a dried form to be used in conjunction with a suitable vehicle such as sterile, pyrogen-free water. The preparation of parenteral formulations under sterile conditions, for example, by lyophilization, may readily be accomplished using standard pharmaceutical techniques well known to those skilled in the art. Parenteral administration of a compound is illustratively performed in the form of saline solutions or with the compound incorporated into liposomes. In cases where the compound in itself is not sufficiently soluble to be dissolved, a solubilizer such as ethanol can be applied.

The dosage of each compound of the claimed combinations depends on several factors, including: the administration method, the condition to be treated, the severity of the condition, whether the condition is to be treated or prevented, and the age, weight, and health of the person to be treated. Additionally, pharmacogenomic (the effect of genotype on the pharmacokinetic, pharmacodynamic or efficacy profile of a therapeutic) information about a particular patient may affect the dosage used.

It is to be understood that in the methods described herein, the individual components of a co-administration, or combination can be administered by any suitable means, contemporaneously, simultaneously, sequentially, separately or in a single pharmaceutical formulation. Where the co-administered compounds or compositions are administered in separate dosage forms, the number of dosages administered per day for each compound may be the same or different. The compounds or compositions may be administered via the same or different routes of administration. The compounds or compositions may be administered according to simultaneous or alternating regimens, at the same or different times during the course of the therapy, concurrently in divided or single forms.

The term “therapeutically effective amount” as used herein, refers to that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated. In one aspect, the therapeutically effective amount is that which may treat or alleviate the disease or symptoms of the disease at a reasonable benefit/risk ratio applicable to any medical treatment. However, it is to be understood that the total daily usage of the compounds and compositions described herein may be decided by the attending physician within the scope of sound medical judgment. The specific therapeutically-effective dose level for any particular patient will depend upon a variety of factors, including the disorder being treated and the severity of the disorder; activity of the specific compound employed; the specific composition employed; the age, body weight, general health, gender and diet of the patient: the time of administration, route of administration, and rate of excretion of the specific compound employed; the duration of the treatment; drugs used in combination or coincidentally with the specific compound employed; and like factors well known to the researcher, veterinarian, medical doctor or other clinician of ordinary skill.

Depending upon the route of administration, a wide range of permissible dosages are contemplated herein, including doses falling in the range from about 1 μg/kg to about 1 g/kg. The dosages may be single or divided, and may administered according to a wide variety of protocols, including q.d. (once a day), b.i.d. (twice a day), t.i.d. (three times a day), or even every other day, once a week, once a month, once a quarter, and the like. In each of these cases it is understood that the therapeutically effective amounts described herein correspond to the instance of administration, or alternatively to the total daily, weekly, month, or quarterly dose, as determined by the dosing protocol.

In addition to the illustrative dosages and dosing protocols described herein, it is to be understood that an effective amount of any one or a mixture of the compounds described herein can be determined by the attending diagnostician or physician by the use of known techniques and/or by observing results obtained under analogous circumstances. In determining the effective amount or dose, a number of factors are considered by the attending diagnostician or physician, including, but not limited to the species of mammal, including human, its size, age, and general health, the specific disease or disorder involved, the degree of or involvement or the severity of the disease or disorder, the response of the individual patient, the particular compound administered, the mode of administration, the bioavailability characteristics of the preparation administered, the dose regimen selected, the use of concomitant medication, and other relevant circumstances.

The term “patient” includes human and non-human animals such as companion animals (dogs and cats and the like) and livestock animals. Livestock animals are animals raised for food production. The patient to be treated is preferably a mammal, in particular a human being.

The α-methylene-γ-butyrolactone (AMGBL, Scheme 1) moiety is present in nearly 10% of all known natural products [7], particularly in a large number of pharmacologically important sesquiterpene lactones [8-11] with a wide range of biological activities. The medicinal properties of these AMGBLs, present in indigenous medicinal herbs, have been attributed to the Michael addition of cytoplasmic thiols to the α,β-unsaturated lactones [12]. Despite the concerns associated with their cytotoxicity, consideration for potential use as actual and pro-drugs in medicinal chemistry is undergoing a recent revival [13]. Seven decades of scant literature reports on the antibacterial activity of natural sesquiterpene AMGBLs, uncharacteristically, show no promise [14-19]. As part of a project involving AMGBLs as NF-κB inhibitors for treating pancreatic cancer, we had introduced novel protocols for the synthesis of a variety of non-natural, substituted AMGBLs [20].

The derivatization of AMGBLs with amines converts them to α-aminomethyl-γ-butyrolactones (AAMLAs, Scheme 1), that are non-toxic, safer pro-drugs with effective drug-like properties [21]. During our project, we not only described the amination of synthetic AMGBLs to AAMLAs [22], but also developed a novel aminolactonization methodology to prepare such AAMLAs directly from α-methylene-γ-hydroxy esters AMGHEs (Scheme 1) [23]. Few literature reports discuss the preparation of AAMLAs from natural sesquiterpene lactones for their anti-cancer properties [24]. AAMLAs have never been examined as antimicrobials until our current project. We reasoned that the investigation of appropriately substituted, synthetic AAMLAs for antibiotic properties could lead to novel antimicrobials.

We have prepared and examined a series of AMGHEs, AMGBLs, and AAMLAs against MRSA and have now discovered that synthetic α-methylene-γ-butyrolactones (AMGBLs, 2), their precursor α-methylene-γ-hydroxy esters (AMGHEs, 1), and their derivatives α-aminomethyl-γ-butyrolactones (AAMLAs, 3) (Scheme 1) can act as new classes of antibiotics with excellent potential to treat MRSA.

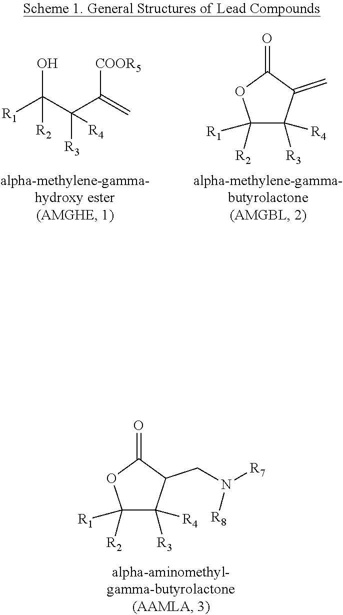

Our assay (Table 1) has revealed that a hydroxy ester (e.g. 148 and 149), the corresponding methylene lactone (e.g. 155 and 158), and aminomethyllactone (e.g. 167) show similar potencies and have potency comparable to vancomycin. Hydroxy esters and the corresponding lactones 144 and 151 show potency similar to the oxazolidinone class of antimicrobial drug linezolid. We have discovered that the substituents at the β- and γ-positions must be an aromatic moiety for the drug to be effective. The results from additional bio-assay of selected AMGHEs, AMGBLs, and AAMLs are described below.

This invention is based on the expectation that synthesis of α-methylenelactones and α-alkylaminolactones, particularly the γ-lactones will provide a novel class of molecules for the treatment of bacterial infection.

A series of γ-hydroxy-α-methylene esters of the general structural formula V were prepared according to published procedures [45-49]. They were converted to the corresponding AMGBL (general structural formula VII) as described earlier. Molecules of formula VII were converted to the corresponding α-aminomethyllactones (general structural formula VI) as described previously [22]. Molecules of formula I were directly converted to molecules VI as described by us [23].

The general procedure of synthesis is shown in Scheme 2 below.

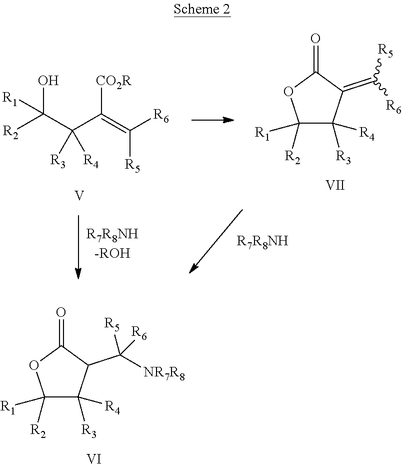

Representative procedures for the synthesis of compounds of formulae V, VI, and VII are given below.

General procedure for preparation of cis-γ-hydroxyester (formula V) [21].

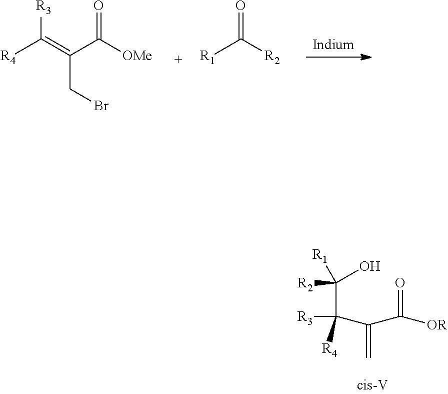

An appropriate aldehyde or ketone (1.5 equiv) were added to a solution of alkyl bromomethacrylate (1.0 eq) in a mixture of THF and water. The solution was rapidly stirred at room temperature and indium powder (1.2 equiv) was added in one portion. The suspension was stirred until the reaction was complete as judged by thin layer chromatographic (TLC) analysis. The mixture was partitioned between water and ethyl acetate, and the organic layer was removed. The crude product was extracted from the aqueous layer one further time with ethyl acetate. The combined organic layers were washed with water then brine, then was dried over Na₂SO₄. After filtration, the product was purified via flash column chromatography to yield the desired cis-γ-hydroxyester.

General procedure for the preparation of trans-lactone (compound formula VII).

(i) General Procedure for the Preparation of 'Crotyl'Boronate [22].

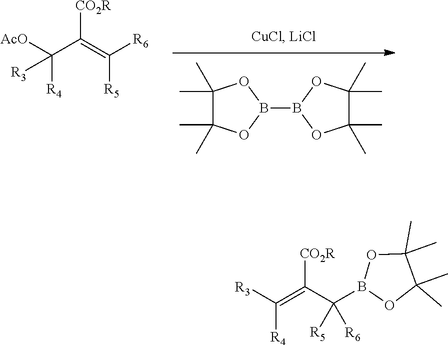

A mixture of CuCl (1.3 equiv) and LiCl (1.3 equiv) in DMF was stirred under N₂ for 1 h at 25° C. and the diboronate (Bis(pinacolato)diboron) (1.3 equiv) dissolved in DMF was added to the reaction mixture. KOAc (1.3 equiv) was added to the reaction mixture followed by the addition of the allylic acetate (1.0 equiv) dissolved in DMF. The reaction mixture was further stirred for 3-5 h. The reaction mixture was then quenched with water and extracted with ether. The combined organic layers were dried over MgSO₄, concentrated under vacuum, and purified by column chromatography to obtain crotylboronates.

(ii) General Procedure for the Preparation of Trans-Lactones [23].

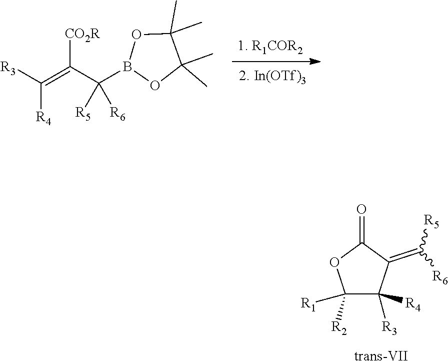

Crotylboronate (1 equiv) was dissolved in toluene and aldehyde (1.2 equiv) was added. The reaction mixture was refluxed for 8-12 h until the completion of the reaction as was monitored by ¹¹B NMR spectroscopy. The reaction mixture was then cooled to room temperature and 20% In(OTf)₃ was added, along with 20% aldehyde and stirred for 2-4 h. The mixture was washed with water and the product was extracted with ether (3×5 mL). The combined organic layer was washed with brine, dried over anhydrous MgSO₄, and concentrated in vacuo. The crude product was purified by column chromatography to obtain the trans-lactones.

Alternative procedure for the preparation of trans-lactone (formula VII) [24].

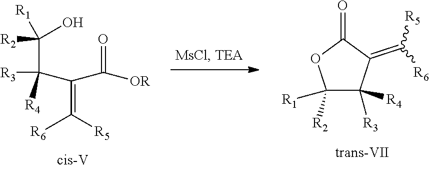

Triethylamine (TEA) (1.1 equiv) was added to a solution of γ-hydroxyester (1.0 equiv) in dichloromethane (DCM). Methanesulfonyl chloride (MsCl) (1.1 equiv) was added drop-wise, and the reaction mixture was stirred until judged complete by TLC analysis. The reaction mixture was quenched with saturated, aqueous ammonium sulfate, then was extracted with DCM. After concentrating the combined organic layers in vacuo, the product was purified via column chromatography to obtain the trans-lactone.

General Procedure for the Preparation of Cis-Lactone (Formula VII) [25].

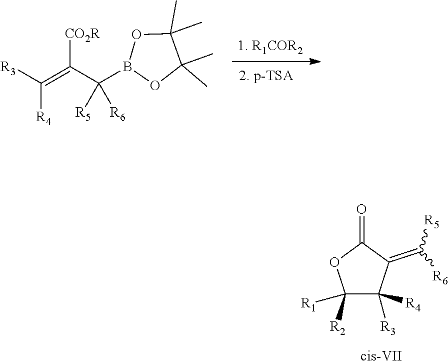

Crotylboronate (1 equiv) was dissolved in toluene and aldehyde (1.2 equiv) was added. The reaction mixture was refluxed for 8-12 h until the completion of the reaction as was monitored by ¹¹B NMR spectroscopy. The reaction mixture was then cooled to room temperature and 20% p-toluenesulfonic acid (p-TSA, p-TsOH) was added, along with 20% aldehyde and stirred for 4-5 h. The mixture was washed with water and the product was extracted with ether. The combined organic layer was washed with brine, dried over anhydrous MgSO₄, and concentrated in vacuo. The crude product was purified by column chromatography to obtain the cis-lactones.

Alternative procedure for the preparation of cis-lactones [20].

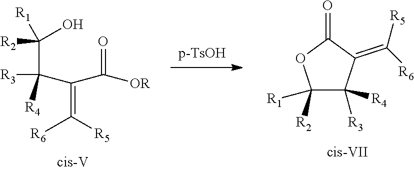

To a solution of cis-γ-hydroxyester (1.0 equiv) in DCM was added p-TSA (0.1 equiv). The solution was stirred until judged as complete by TLC. After partitioning between water and dichloromethane, the organic layer was dried over Na₂SO₄. The crude product was purified by column chromatography to obtain the desired cis-lactone.

General Procedure for the Preparation of the Aminolactone Via Conjugate Addition to VII (Compound Formula VI) [26].

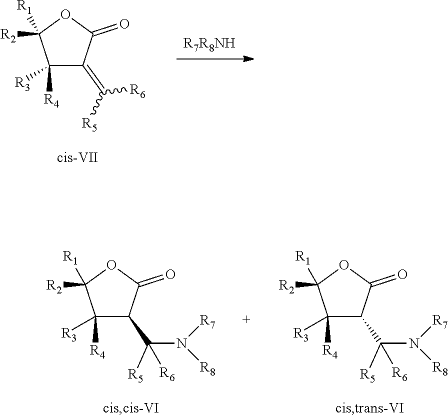

To a solution of cis-α-methylenelactone (1.0 equiv) in DCM was added N,N-dialkylamine (3.0 equiv) in one portion at room temperature. The solution was rapidly stirred until the reaction was complete as judged by TLC analysis. The crude mixture was directly concentrated in vacuo to remove the solvent and excess amine. If deemed necessary, the crude reaction mixture was purified by flash column chromatography with amine additive to yield the desired aminolactone.

General Procedure for Aminolactonization. Direct Synthesis of Aminolactones (Compound Formula VI) from γ-Hydroxyesters (Compound Formula V) [27].

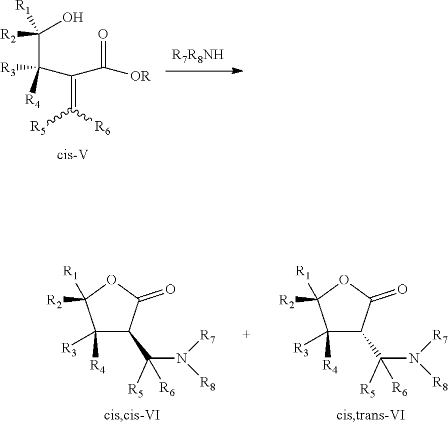

To a solution of cis-γ-hydroxyester (1.0 equiv) in DCM was added N,N-dialkylamine (3.0 equiv) in one portion at room temperature. The solution was rapidly stirred until the reaction was complete as judged by TLC analysis. The crude mixture was directly concentrated in vacuo to remove the solvent and excess amine. If deemed necessary, the crude reaction mixture was purified by flash column chromatography with amine additive to yield the desired aminolactone.

In Vitro Bio-Assays

Initial Screening of Compounds Against Methicillin Resistant Staphylococcus aureus NRS 384 (USA300)

Method: The minimum inhibitory concentration (MIC) of the compounds 1-187, and control antibiotics (vancomycin, linezolid) was determined using the broth microdilution method against methicillin-resistant Staphylococcus aureus (MRSA) USA 300 using the broth microdilution method according to the guidelines outlined by the Clinical and Laboratory Standards Institute (CLSI) [25]. MRSA strain was cultivated on Tryptic soy agar plates and let grow for 24 hours at 37° C. under aerobic conditions. Bacterial suspension equivalent to 0.5 McFarland standard was prepared and diluted in cation-adjusted Mueller-Hinton broth (CAMHB) to attain a bacterial concentration of 5×10⁵ CFU/ml and seeded in 96-well plates. The desired concentrations of each drug and compound were added in the first row of the 96-well plates and serially diluted to achieve a concentration gradient from 64 μg/ml to 0.5 μg/mL. MICs reported in Table 1 are the concentrations of compounds/control drugs that have been found to completely inhibit the bacterial growth when determined visually.

TABLE 1
Minimum Inhibitory Concentration (MIC in μg/mL) of AMGHEs,
AMGBLs, AAMLAs and AAMLA fumarate salts and control drugs screened against
Staphylococcus aureus NRS384 (MRSA USA300)

| | | MIC | | | |
|---|---|---|---|---|---|
| # | Compound | (μg/mL) | Mol. Type | R₁ | R₂ |
| 1 | 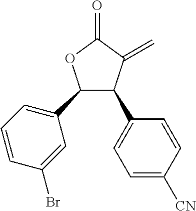 [SMILES: C=C1C(=O)O[C@H](c2cccc(Br)c2)[C@@H]1c1ccc(C#N)cc1] | 8 | Methylene Cis- lactone | p-cyano | m-bromo |
| 2 | 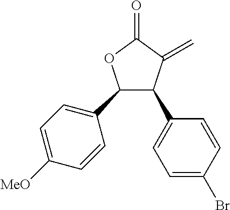 [SMILES: C=C1C(=O)O[C@H](c2ccc(OC)cc2)[C@@H]1c1ccc(Br)cc1] | 8 | Methylene Cis- lactone | p-methoxy | p-bromo |
| 3 | 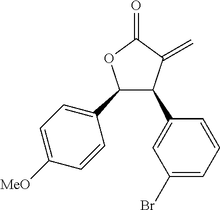 [SMILES: C=C1C(=O)O[C@H](c2ccc(OC)cc2)[C@@H]1c1cccc(Br)c1] | 8 | Methylene Cis- lactone | p-methoxy | m-bromo |
| 4 | 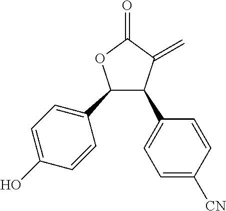 [SMILES: C=C1C(=O)O[C@H](c2ccc(O)cc2)[C@@H]1c1ccc(C#N)cc1] | 256 | Methylene Cis- lactone | p-hydroxy | p-cyano |
| 5 | 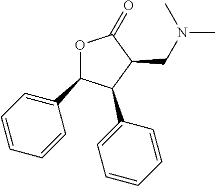 [SMILES: CN(C)C[C@@H]1C(=O)O[C@H](c2ccccc2)[C@@H]1c1ccccc1] | 16 | Dimethylamino Cis-Lactone | phenyl | phenyl |
| 6 | 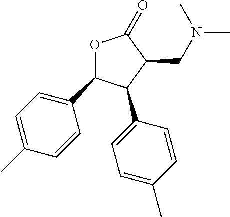 | 32 | Dimethylamino Cis-Lactone | p-methyl | p-methyl |
| 7 | 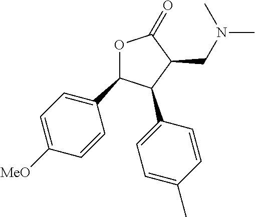 [SMILES: COc1ccc([C@H]2OC(=O)[C@@H](CN(C)C)[C@H]2c2ccc(C)cc2)cc1] | 32 | Dimethylamino Cis-Lactone | p-methyl | p-methoxy |
| 8 | 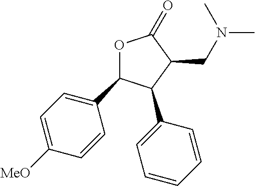 [SMILES: COc1ccc([C@H]2OC(=O)[C@@H](CN(C)C)[C@H]2c2ccccc2)cc1] | >256 | Dimethylamino Cis-Lactone | phenyl | p-methoxy |
| 9 | 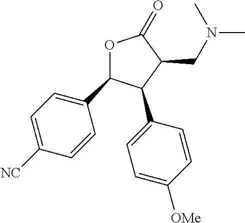 [SMILES: COc1ccc([C@@H]2[C@H](CN(C)C)C(=O)O[C@@H]2c2ccc(C#N)cc2)cc1] | 128 | Dimethylamino Cis-Lactone | p-methoxy | p-cyano |
| 10 | 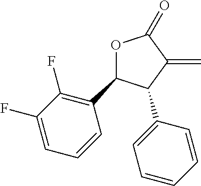 [SMILES: C=C1C(=O)O[C@H](c2cccc(F)c2F)[C@H]1c1ccccc1] | 16 | Methylene Trans-lactone | phenyl | 2,3-difluoro |
| 11 | 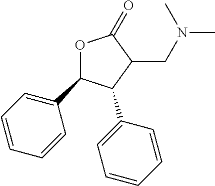 [SMILES: CN(C)CC1C(=O)O[C@H](c2ccccc2)[C@H]1c1ccccc1] | 32 | Dimethylamino Trans-Lactone | phenyl | phenyl |
| 12 | 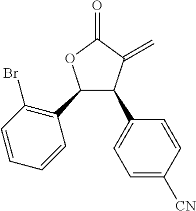 [SMILES: C=C1C(=O)O[C@H](c2ccccc2Br)[C@@H]1c1ccc(C#N)cc1] | 4 | Methylene lactone | p-cyano | o-bromo |
| 13 | 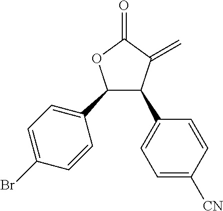 [SMILES: C=C1C(=O)O[C@H](c2ccc(Br)cc2)[C@@H]1c1ccc(C#N)cc1] | 4 | Methylene lactone | p-cyano | p-bromo |
| 14 | 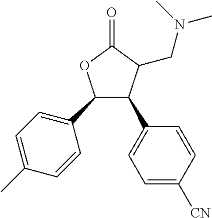 | >128 | Dimethylamino Lactone | p-cyano | p-methyl |
| 15 | 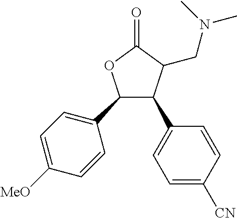 [SMILES: COc1ccc([C@H]2OC(=O)C(CN(C)C)[C@H]2c2ccc(C#N)cc2)cc1] | >128 | Dimethylamino Lactone | p-cyano | p-methoxy |
| 16 | 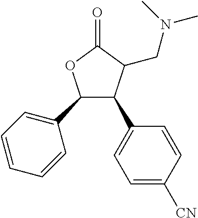 [SMILES: CN(C)CC1C(=O)O[C@H](c2ccccc2)[C@@H]1c1ccc(C#N)cc1] | >128 | Dimethylamino Lactone | p-cyano | phenyl |
| 17 | 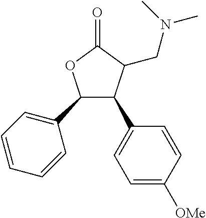 [SMILES: COc1ccc([C@@H]2C(CN(C)C)C(=O)O[C@@H]2c2ccccc2)cc1] | 64 | Dimethylamino Lactone | p-methoxy | phenyl |
| 18 | 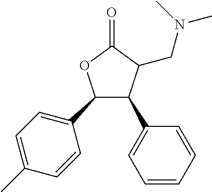 | 128 | Dimethylamino Lactone | phenyl | p-methyl |
| 19 | 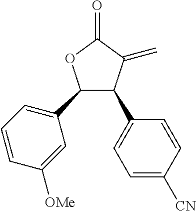 [SMILES: C=C1C(=O)O[C@H](c2cccc(OC)c2)[C@@H]1c1ccc(C#N)cc1] | 64 | Methylene lactone | p-cyano | m-methyl |
| 20 | 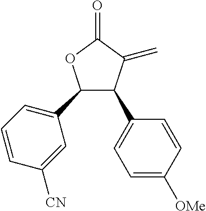 [SMILES: C=C1C(=O)O[C@H](c2cccc(C#N)c2)[C@@H]1c1ccc(OC)cc1] | 16 | Methylene lactone | p-methoxy | m-cyano |
| 21 | 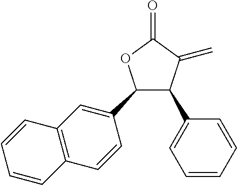 [SMILES: C=C1C(=O)O[C@H](c2ccc3ccccc3c2)[C@@H]1c1ccccc1] | 32 | Methylene lactone | phenyl | 2-naphthyl |
| 22 | 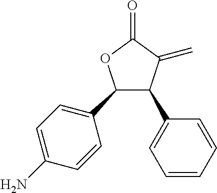 [SMILES: C=C1C(=O)O[C@H](c2ccc(N)cc2)[C@@H]1c1ccccc1] | 32 | Methylene lactone | phenyl | p-amino |
| 23 | 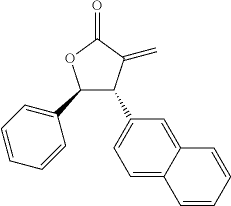 [SMILES: C=C1C(=O)O[C@H](c2ccccc2)[C@H]1c1ccc2ccccc2c1] | 2 | Methylene lactone | 2-naphthyl | phenyl |
| 24 | 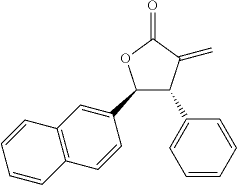 [SMILES: C=C1C(=O)O[C@H](c2ccc3ccccc3c2)[C@H]1c1ccccc1] | 8 | Methylene lactone | phenyl | 2-naphthyl |
| 25 | 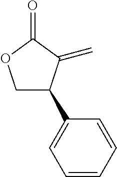 | >128 | Methylene lactone | phenyl | hydrogen |
| 26 | 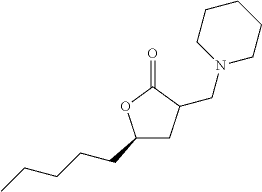 [SMILES: CCCCC[C@@H]1CC(CN2CCCCC2)C(=O)O1] | >128 | Piperidino lactone | hydrogen | n-pentyl |
| 27 | 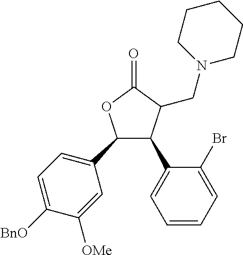 [SMILES: COc1cc([C@H]2OC(=O)C(CN3CCCCC3)[C@H]2c2ccccc2Br)ccc1OCc1ccccc1] | >128 | Piperidino lactone | o-bromo | m-methoxy, p- benzyloxy |
| 28 | 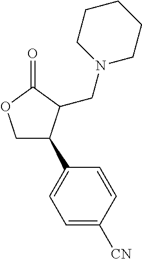 [SMILES: N#Cc1ccc([C@H]2COC(=O)C2CN2CCCCC2)cc1] | >128 | Piperidino lactone | p-cyano | hydrogen |
| 29 | 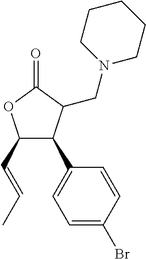 [SMILES: C/C=C/[C@H]1OC(=O)C(CN2CCCCC2)[C@H]1c1ccc(Br)cc1] | 16 | Piperidino lactone | p-bromo | |
| 30 | 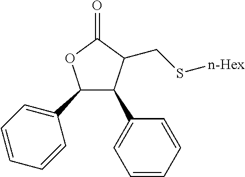 | >128 | | phenyl | phenyl |
| 31 | 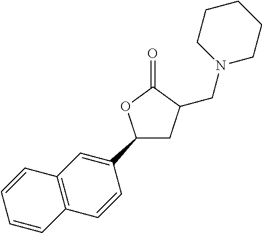 [SMILES: O=C1O[C@H](c2ccc3ccccc3c2)CC1CN1CCCCC1] | >128 | Piperidino lactone | hydrogen | 2-naphthyl |
| 32 | 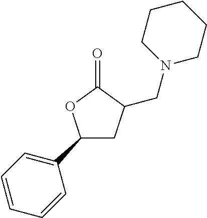 [SMILES: O=C1O[C@H](c2ccccc2)CC1CN1CCCCC1] | >128 | Piperidino lactone | hydrogen | phenyl |
| 33 | 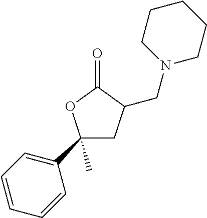 [SMILES: C[C@@]1(c2ccccc2)CC(CN2CCCCC2)C(=O)O1] | >128 | Piperidino lactone | hydrogen | |
| 34 | 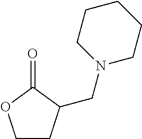 [SMILES: O=C1OCCC1CN1CCCCC1] | >128 | Piperidino lactone | hydrogen | hydrogen |
| 35 | 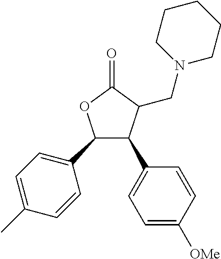 [SMILES: COc1ccc([C@@H]2C(CN3CCCCC3)C(=O)O[C@@H]2c2ccc(C)cc2)cc1] | 64 | Piperidino lactone | p-methoxy | p-methyl |
| 36 | 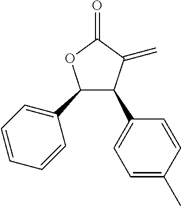 [SMILES: C=C1C(=O)O[C@H](c2ccccc2)[C@@H]1c1ccc(C)cc1] | 32 | Methylene lactone | p-methyl | phenyl |
| 37 | 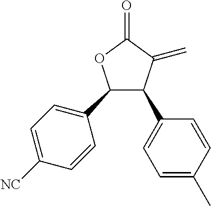 [SMILES: C=C1C(=O)O[C@H](c2ccc(C#N)cc2)[C@@H]1c1ccc(C)cc1] | 16 | Methylene lactone | p-methyl | p-cyano |
| 38 | 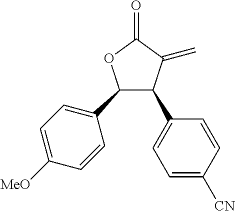 [SMILES: C=C1C(=O)O[C@H](c2ccc(OC)cc2)[C@@H]1c1ccc(C#N)cc1] | 32 | Methylene lactone | p-cyano | p-methoxy |
| 39 | 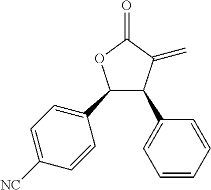 [SMILES: C=C1C(=O)O[C@H](c2ccc(C#N)cc2)[C@@H]1c1ccccc1] | 64 | Methylene lactone | phenyl | p-cyano |
| 40 | 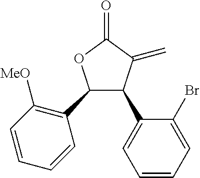 [SMILES: C=C1C(=O)O[C@H](c2ccccc2OC)[C@@H]1c1ccccc1Br] | 16 | Methylene lactone | o-bromo | o-methoxy |
| 41 | 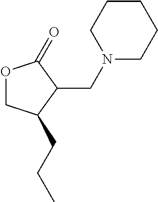 [SMILES: CCC[C@H]1COC(=O)C1CN1CCCCC1] | >128 | Piperidino lactone | n-propyl | hydrogen |
| 42 | 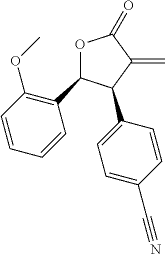 [SMILES: C=C1C(=O)O[C@H](c2ccccc2OC)[C@@H]1c1ccc(C#N)cc1] | 32 | Methylene lactone | p-cyano | o-methoxy |
| 43 | 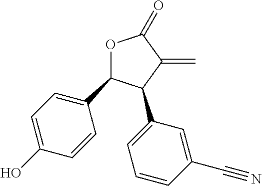 [SMILES: C=C1C(=O)O[C@H](c2ccc(O)cc2)[C@@H]1c1cccc(C#N)c1] | 128 | Methylene lactone | m-cyano | p-hydroxy |
| 44 | 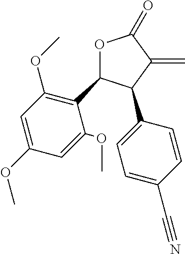 [SMILES: C=C1C(=O)O[C@H](c2c(OC)cc(OC)cc2OC)[C@@H]1c1ccc(C#N)cc1] | >128 | Methylene lactone | p-cyano | 2,4,6- trimethoxy |
| 45 | 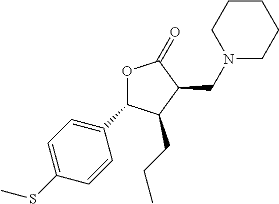 [SMILES: CCC[C@@H]1[C@H](CN2CCCCC2)C(=O)O[C@H]1c1ccc(SC)cc1] | >128 | Piperidino lactone | n-propyl | p-methoxy |
| 46 | 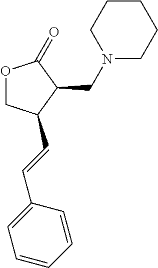 [SMILES: O=C1OC[C@H](/C=C/c2ccccc2)[C@@H]1CN1CCCCC1] | >128 | Piperidino lactone | | hydrogen |
| 47 | 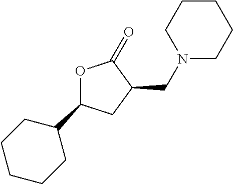 [SMILES: O=C1O[C@H](C2CCCCC2)C[C@@H]1CN1CCCCC1] | >128 | Piperidino lactone | hydrogen | cyclohexyl |
| 48 | 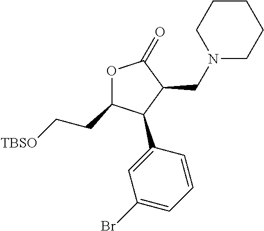 [SMILES: CC(C)(C)[Si](C)(C)OCC[C@H]1OC(=O)[C@@H](CN2CCCCC2)[C@H]1c1cccc(Br)c1] | >128 | Piperidino lactone | m-bromo | |
| 49 | 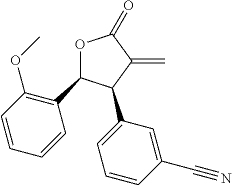 [SMILES: C=C1C(=O)O[C@H](c2ccccc2OC)[C@@H]1c1cccc(C#N)c1] | 16 | Methylene lactone | m-cyano | o-methoxy |
| 50 | 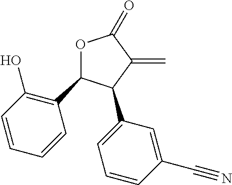 [SMILES: C=C1C(=O)O[C@H](c2ccccc2O)[C@@H]1c1cccc(C#N)c1] | >128 | Methylene lactone | m-cyano | o-hydroxy |
| 51 | 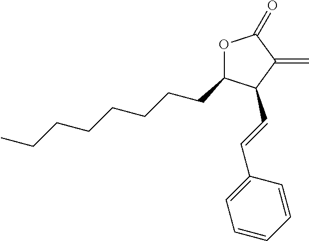 [SMILES: C=C1C(=O)O[C@H](CCCCCCCC)[C@@H]1/C=C/c1ccccc1] | >128 | Methylene lactone | | n-octyl |
| 52 | 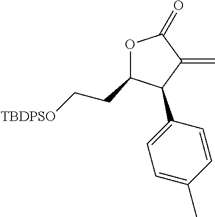 | >128 | Methylene lactone | p-methyl | |
| 53 | 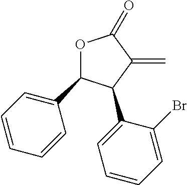 [SMILES: C=C1C(=O)O[C@H](c2ccccc2)[C@@H]1c1ccccc1Br] | 4 | Methylene lactone | o-bromo | Phenyl |
| 54 | 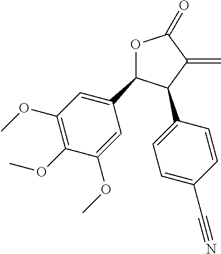 | 128 | Methylene lactone | p-cyano | 3,4,5- trimethoxy |
| 55 | 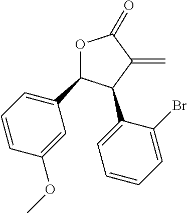 [SMILES: C=C1C(=O)O[C@H](c2cccc(OC)c2)[C@@H]1c1ccccc1Br] | 8 | Methylene lactone | o-bromo | m-methoxy |
| 56 | 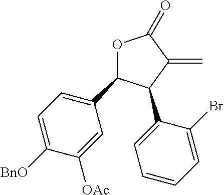 [SMILES: C=C1C(=O)O[C@H](c2ccc(OCc3ccccc3)c(OC(C)=O)c2)[C@@H]1c1ccccc1Br] | >128 | Methylene lactone | o-bromo | m-methoxy, p- benzyloxy |
| 57 | 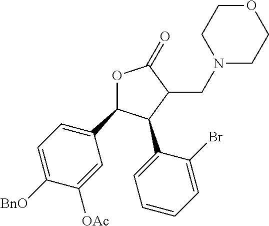 [SMILES: CC(=O)Oc1cc([C@H]2OC(=O)C(CN3CCOCC3)[C@H]2c2ccccc2Br)ccc1OCc1ccccc1] | >128 | Piperidino lactone | o-bromo | m-methoxy, p- benzyloxy |
| 58 | 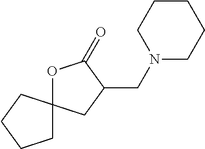 [SMILES: O=C1OC2(CCCC2)CC1CN1CCCCC1] | >128 | Piperidino lactone | hydrogen | |
| 59 | 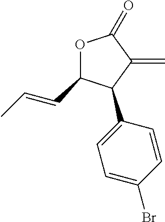 [SMILES: C=C1C(=O)O[C@H](/C=C/C)[C@@H]1c1ccc(Br)cc1] | 32 | Methylene lactone | p-bromo | |
| 61 | 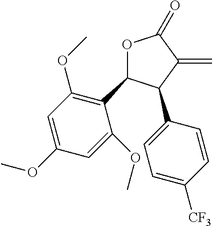 | >128 | Methylene lactone | p- trifluoromethyl | 2,4,6- trimethoxy |
| 62 | 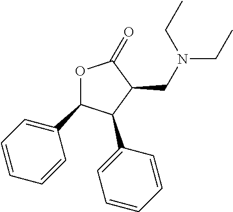 [SMILES: CCN(CC)C[C@@H]1C(=O)O[C@H](c2ccccc2)[C@@H]1c1ccccc1] | 16 | Diethylamino lactone | Phenyl | Phenyl |
| 63 | 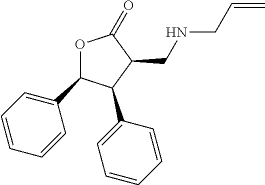 [SMILES: C=CCNC[C@@H]1C(=O)O[C@H](c2ccccc2)[C@@H]1c1ccccc1] | 128 | Allylamino lactone | Phenyl | Phenyl |
| 64 | 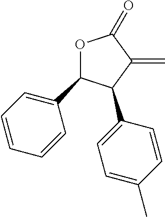 [SMILES: C=C1C(=O)O[C@H](c2ccccc2)[C@@H]1c1ccc(C)cc1] | 32 | Methylene lactone | p-methyl | Phenyl |
| 65 | 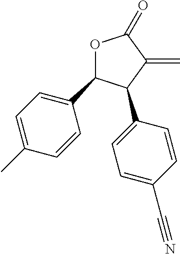 | 4 | Methylene lactone | p-cyano | p-methyl |
| 66 | 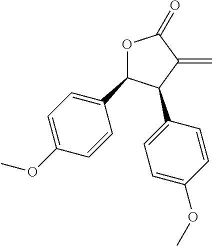 | 32 | Methylene lactone | p-methoxy | p-methoxy |
| 67 | 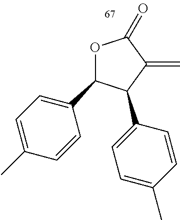 | 64 | Methylene lactone | p-methyl | p-methyl |
| 68 | 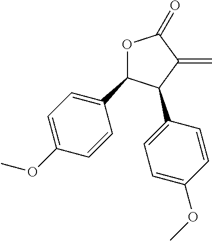 | 64 | Methylene lactone | p-methoxy | p-methoxy |
| 69 | 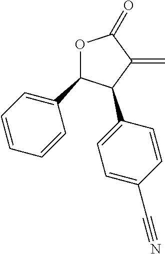 [SMILES: C=C1C(=O)O[C@H](c2ccccc2)[C@@H]1c1ccc(C#N)cc1] | >128 | Methylene lactone | p-cyano | Phenyl |
| 70 | 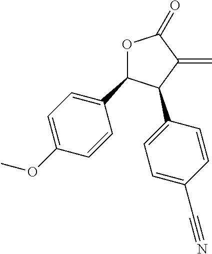 | 32 | Methylene lactone | p-cyano | p-methoxy |
| 71 | 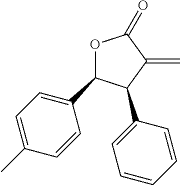 | 32 | Methylene lactone | Phenyl | p-methyl |
| 72 | 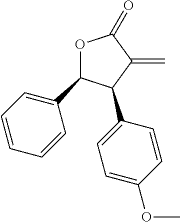 [SMILES: C=C1C(=O)O[C@H](c2ccccc2)[C@@H]1c1ccc(OC)cc1] | 64 | Methylene lactone | p-methoxy | Phenyl |
| 74 | 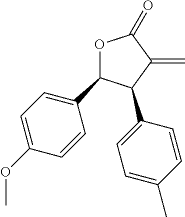 [SMILES: C=C1C(=O)O[C@H](c2ccc(OC)cc2)[C@@H]1c1ccc(C)cc1] | 16 | Methylene lactone | p-methyl | p-methoxy |
| 75 | 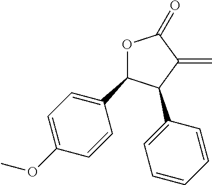 | 64 | Methylene lactone | Phenyl | p-methoxy |
| 76 | 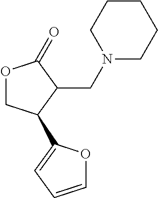 [SMILES: O=C1OC[C@H](c2ccco2)C1CN1CCCCC1] | >128 | Methylene lactone | | |
| 77 | 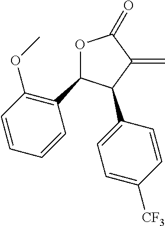 [SMILES: C=C1C(=O)O[C@H](c2ccccc2OC)[C@@H]1c1ccc(C(F)(F)F)cc1] | 8 | Methylene lactone | p- trifluoromethyl | o-methoxy |
| 78 | 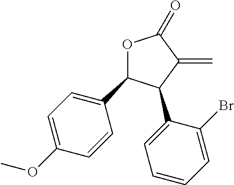 [SMILES: C=C1C(=O)O[C@H](c2ccc(OC)cc2)[C@@H]1c1ccccc1Br] | 8 | Methylene lactone | o-bromo | p-methoxy |
| 79 | 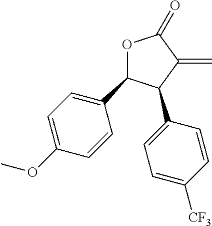 | 8 | Methylene lactone | p- trifluoromethyl | p-methoxy |
| 80 | 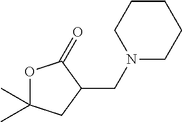 | >128 | Piperidino lactone | hydrogen | |
| 82 | 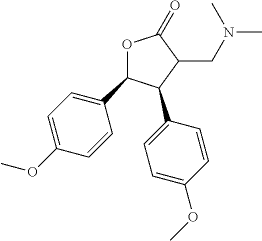 | >128 | Dimethylamino Lactone | p-methoxy | p-methoxy |
| 83 | 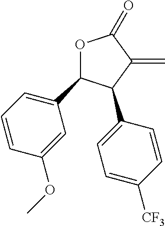 [SMILES: C=C1C(=O)O[C@H](c2cccc(OC)c2)[C@@H]1c1ccc(C(F)(F)F)cc1] | 16 | Methylene lactone | p- trifluoromethyl | m-methoxy |
| 84 | 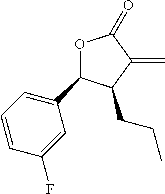 [SMILES: C=C1C(=O)O[C@H](c2cccc(F)c2)[C@@H]1CCC] | >128 | Methylene lactone | n-propyl | m-fluoro |
| 85 | 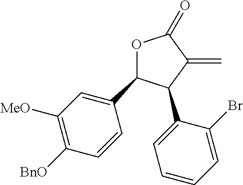 [SMILES: C=C1C(=O)O[C@H](c2ccc(OCc3ccccc3)c(OC)c2)[C@@H]1c1ccccc1Br] | >128 | Methylene lactone | o-bromo | o-methoxy, m- benzyloxy |
| 86 | 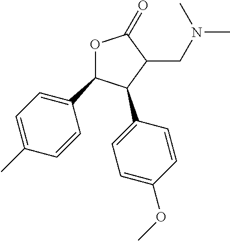 | 128 | Dimethylamino Lactone | p-methoxy | p-methyl |
| 87 | 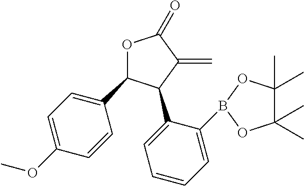 | >128 | Methylene lactone | | p-methoxy |
| 88 | 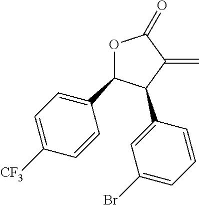 [SMILES: C=C1C(=O)O[C@H](c2ccc(C(F)(F)F)cc2)[C@@H]1c1cccc(Br)c1] | 16 | Methylene lactone | m-bromo | p- trifluoromethyl |
| 90 | 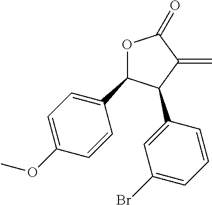 | 8 | Methylene lactone | m-bromo | p-methoxy |
| 91 | 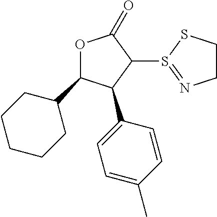 [SMILES: Cc1ccc([C@@H]2C(S3=NCCS3)C(=O)O[C@@H]2C2CCCCC2)cc1] | >128 | | p-methyl | cyclohexyl |
| 92 | 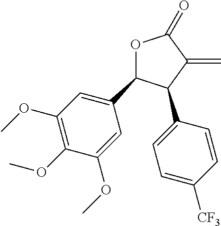 | 32 | Methylene lactone | p- trifluoromethyl | 3,4,5- trimethoxy |
| 93 | 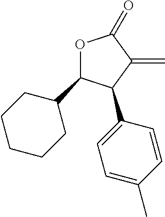 [SMILES: C=C1C(=O)O[C@H](C2CCCCC2)[C@@H]1c1ccc(C)cc1] | >128 | Methylene lactone | p-methyl | cyclohexyl |
| 94 | 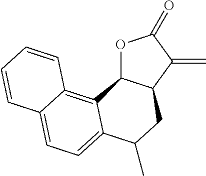 | 32 | Methylene lactone | n-propyl | |
| 95 | 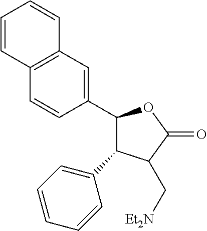 | >128 | Diethylamino Lactone | Phenyl | 2-naphthyl |
| 96 | 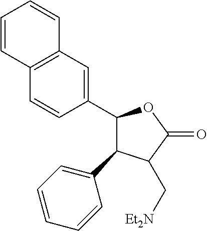 | >128 | Diethylamino Lactone | Phenyl | 2-naphthyl |
| 97 | 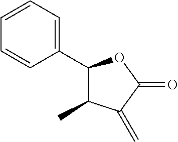 | 16 | Methylene lactone | | Phenyl |
| 98 | 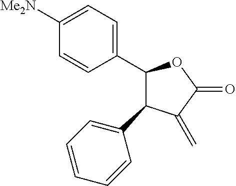 [SMILES: C=C1C(=O)O[C@H](c2ccc(N(C)C)cc2)[C@@H]1c1ccccc1] | >128 | Methylene lactone | Phenyl | p- dimethylamino |
| 99 | 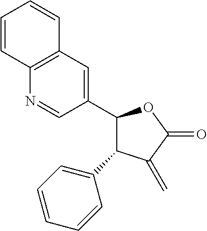 [SMILES: C=C1C(=O)O[C@H](c2cnc3ccccc3c2)[C@H]1c1ccccc1] | 32 | Methylene lactone | Phenyl | |
| 100 | 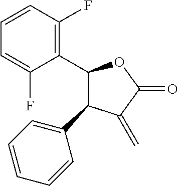 [SMILES: C=C1C(=O)O[C@H](c2c(F)cccc2F)[C@@H]1c1ccccc1] | 8 | Methylene lactone | Phenyl | 2,6-difluoro |
| 101 | 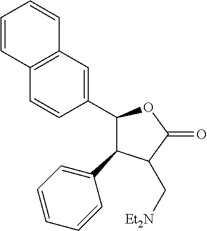 | >128 | Diethylamino Lactone | Phenyl | 2-naphthyl |
| 102 | 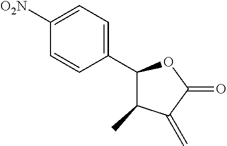 [SMILES: C=C1C(=O)O[C@H](c2ccc([N+](=O)[O-])cc2)[C@@H]1C] | >128 | Methylene lactone | methyl | p-nitro |
| 103 | 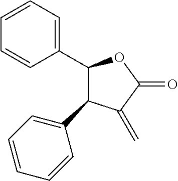 [SMILES: C=C1C(=O)O[C@H](c2ccccc2)[C@@H]1c1ccccc1] | 32 | Methylene lactone | Phenyl | Phenyl |
| 104 | 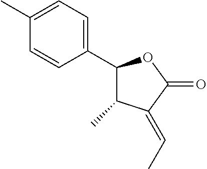 [SMILES: C/C=C1/C(=O)O[C@H](c2ccc(C)cc2)[C@H]1C] | >128 | | methyl | p-methyl |
| 105 | 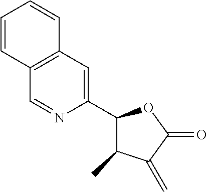 [SMILES: C=C1C(=O)O[C@H](c2cc3ccccc3cn2)[C@@H]1C] | 16 | Methylene lactone | methyl | |
| 106 | 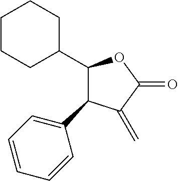 [SMILES: C=C1C(=O)O[C@H](C2CCCCC2)[C@@H]1c1ccccc1] | >128 | Methylene lactone | Phenyl | cyclohexyl |
| 107 | 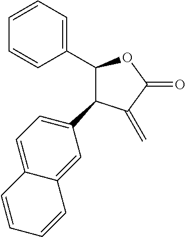 [SMILES: C=C1C(=O)O[C@H](c2ccccc2)[C@@H]1c1ccc2ccccc2c1] | 8 | | 2-naphthyl | Phenyl |
| 108 | 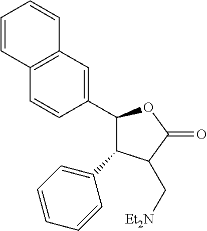 | >128 | Diethylamino Lactone | Phenyl | 2-naphthyl |
| 109 | 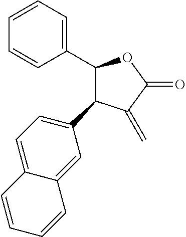 [SMILES: C=C1C(=O)O[C@H](c2ccccc2)[C@@H]1c1ccc2ccccc2c1] | ≤1 | Methylene lactone | 2-naphthyl | Phenyl |
| 110 | 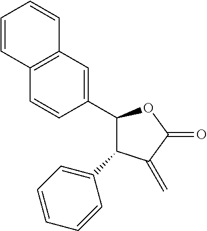 [SMILES: C=C1C(=O)O[C@H](c2ccc3ccccc3c2)[C@H]1c1ccccc1] | 8 | | Phenyl | 2-naphthyl |
| 111 | 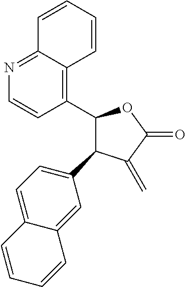 [SMILES: C=C1C(=O)O[C@H](c2ccnc3ccccc23)[C@@H]1c1ccc2ccccc2c1] | 2 | Methylene lactone | 2-naphthyl | |
| 112 | 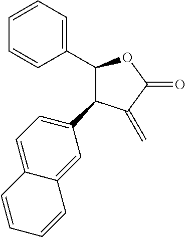 [SMILES: C=C1C(=O)O[C@H](c2ccccc2)[C@@H]1c1ccc2ccccc2c1] | 64 | Methylene lactone | 2-naphthyl | Phenyl |
| 113 | 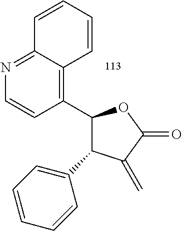 [SMILES: C=C1C(=O)O[C@H](c2ccnc3ccccc23)[C@H]1c1ccccc1] | 16 | Methylene lactone | Phenyl | |
| 114 | 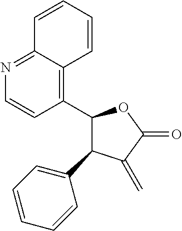 [SMILES: C=C1C(=O)O[C@H](c2ccnc3ccccc23)[C@@H]1c1ccccc1] | 32 | Methylene lactone | Phenyl | |
| 115 | 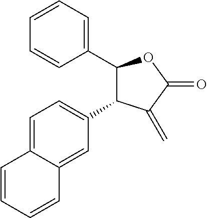 [SMILES: C=C1C(=O)O[C@H](c2ccccc2)[C@H]1c1ccc2ccccc2c1] | 2 | Methylene lactone | 2-naphthyl | Phenyl |
| 116 | 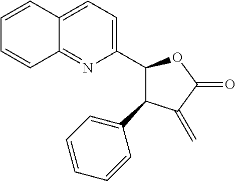 [SMILES: C=C1C(=O)O[C@H](c2ccc3ccccc3n2)[C@@H]1c1ccccc1] | >128 | Methylene lactone | Phenyl | |
| 117 | 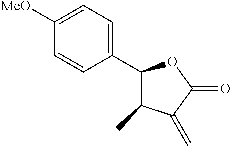 [SMILES: C=C1C(=O)O[C@H](c2ccc(OC)cc2)[C@@H]1C] | >128 | Methylene lactone | methyl | p-methoxy |
| 118 | 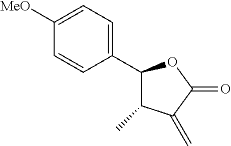 [SMILES: C=C1C(=O)O[C@H](c2ccc(OC)cc2)[C@H]1C] | >128 | Methylene lactone | methyl | p-methoxy |
| 119 | 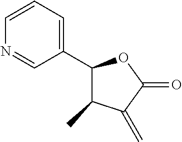 | >128 | Methylene lactone | methyl | |
| 120 | 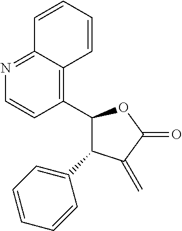 [SMILES: C=C1C(=O)O[C@H](c2ccnc3ccccc23)[C@H]1c1ccccc1] | 128 | Methylene lactone | Phenyl | |
| 121 | 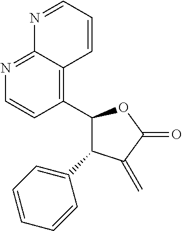 [SMILES: C=C1C(=O)O[C@H](c2ccnc3ncccc23)[C@H]1c1ccccc1] | 32 | Methylene lactone | Phenyl | |
| 123 | | 8 | Methylene | Phenyl | |
| | | | lactone | | |
| 124 | | 64 | Methylene | Phenyl | 2-naphthyl |
| | | | lactone | | |
| 125 | 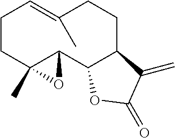 [SMILES: C=C1C(=O)O[C@H]2[C@H]1CC/C(C)=C/CC[C@@]1(C)O[C@@H]21] | 128 | Parthenolide Methylene lactone | | |
| 129 | | >256 | Hydroxy Ester | | |
| 130 | | >256 | Piperidino | | |
| | | | Lactone | | |
| 131 | | >256 | Hydroxy Ester | | |
| 132 | | >256 | Piperidino | | |
| | | | Lactone | | |
| | | | Fumarate | | |
| 135 | | >256 | Hydroxy Ester | | |
| 136 | | >256 | Hydroxy Ester | | |
| 137 | | >256 | Piperidino | | |
| | | | Lactone | | |
| 138 | | >256 | Piperidino | | |
| | | | Lactone | | |
| 139 | | >256 | Piperidino | | |
| | | | Lactone | | |
| | | | Fumarate | | |
| 140 | | >256 | Piperidino | | |
| | | | Lactone | | |
| | | | Fumarate | | |
| 141 | 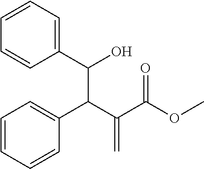 [SMILES: C=C(C(=O)OC)C(c1ccccc1)C(O)c1ccccc1] | 16 | Hydroxy Ester | Phenyl | Phenyl |
| 142 | 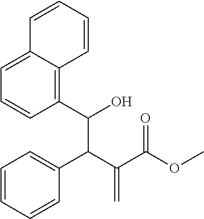 [SMILES: C=C(C(=O)OC)C(c1ccccc1)C(O)c1cccc2ccccc12] | 4 | Hydroxy Ester | Phenyl | 1-naphthyl |
| 143 | 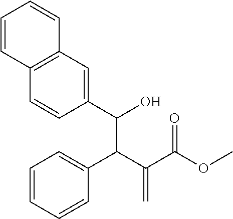 [SMILES: C=C(C(=O)OC)C(c1ccccc1)C(O)c1ccc2ccccc2c1] | 16 | Hydroxy Ester | Phenyl | 2-naphthyl |
| 144 | 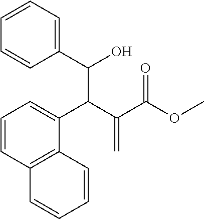 [SMILES: C=C(C(=O)OC)C(c1cccc2ccccc12)C(O)c1ccccc1] | 1 | Hydroxy Ester | 1-naphthyl | Phenyl |
| 145 | 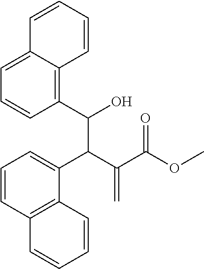 [SMILES: C=C(C(=O)OC)C(c1cccc2ccccc12)C(O)c1cccc2ccccc12] | >64 | Hydroxy Ester | 1-naphthyl | 1-naphthyl |
| 146 | 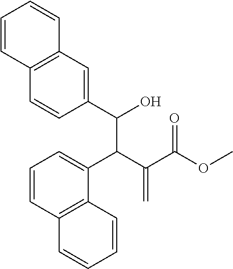 [SMILES: C=C(C(=O)OC)C(c1cccc2ccccc12)C(O)c1ccc2ccccc2c1] | >64 | Hydroxy Ester | 1-naphthyl | 2-naphthyl |
| 147 | 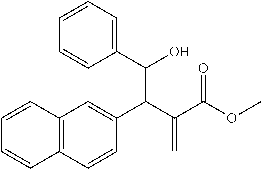 [SMILES: C=C(C(=O)OC)C(c1ccc2ccccc2c1)C(O)c1ccccc1] | 8 | Hydroxy Ester | 2-naphthyl | Phenyl |
| 148 | 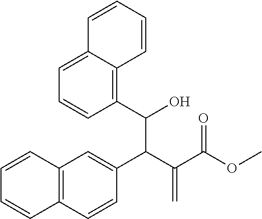 [SMILES: C=C(C(=O)OC)C(c1ccc2ccccc2c1)C(O)c1cccc2ccccc12] | 2 | Hydroxy Ester | 2-naphthyl | 1-naphthyl |
| 149 | 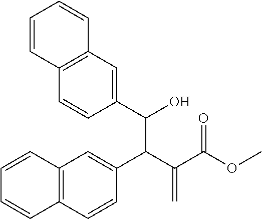 [SMILES: C=C(C(=O)OC)C(c1ccc2ccccc2c1)C(O)c1ccc2ccccc2c1] | 2 | Hydroxy Ester | 2-naphthyl | 2-naphthyl |
| 150 | 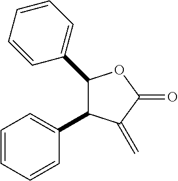 [SMILES: C=C1C(=O)O[C@H](c2ccccc2)[C@@H]1c1ccccc1] | 16 | Methylene Lactone | Phenyl | Phenyl |
| 151 | 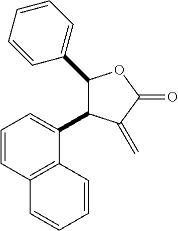 [SMILES: C=C1C(=O)O[C@H](c2ccccc2)[C@@H]1c1cccc2ccccc12] | 1 | Methylene Lactone | 1-naphthyl | Phenyl |
| 152 | 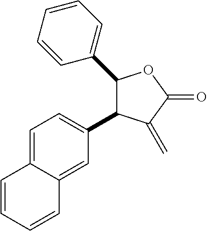 [SMILES: C=C1C(=O)O[C@H](c2ccccc2)[C@@H]1c1ccc2ccccc2c1] | 8 | Methylene Lactone | 2-naphthyl | Phenyl |
| 153 | 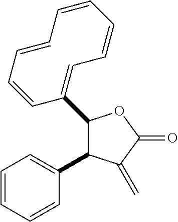 [SMILES: C=C1C(=O)O[C@H](c2ccccccccc2)[C@@H]1c1ccccc1] | 4 | Methylene Lactone | Phenyl | 1-naphthyl |
| 154 | 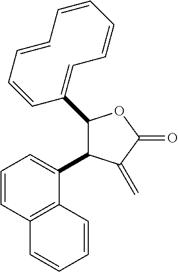 [SMILES: C=C1C(=O)O[C@H](c2ccccccccc2)[C@@H]1c1cccc2ccccc12] | >64 | Methylene Cis- Lactone | 1-naphthyl | 1-naphthyl |
| 155 | 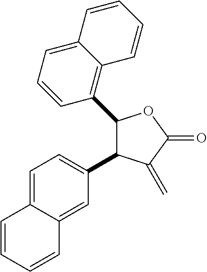 [SMILES: C=C1C(=O)O[C@H](c2cccc3ccccc23)[C@@H]1c1ccc2ccccc2c1] | 2 | Methylene Cis- Lactone | 2-naphthyl | 1-naphthyl |
| 156 | 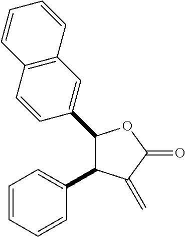 [SMILES: C=C1C(=O)O[C@H](c2ccc3ccccc3c2)[C@@H]1c1ccccc1] | 16 | Methylene Cis- Lactone | Phenyl | 2-naphthyl |
| 157 | 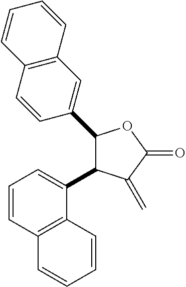 [SMILES: C=C1C(=O)O[C@H](c2ccc3ccccc3c2)[C@@H]1c1cccc2ccccc12] | >64 | Methylene Cis- Lactone | 1-naphthyl | 2-naphthyl |
| 158 | 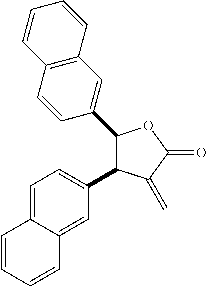 [SMILES: C=C1C(=O)O[C@H](c2ccc3ccccc3c2)[C@@H]1c1ccc2ccccc2c1] | 2 | Methylene Cis- Lactone | 2-naphthyl | 2-naphthyl |
| 159 | 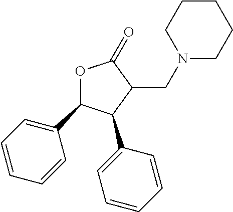 [SMILES: O=C1O[C@H](c2ccccc2)[C@H](c2ccccc2)C1CN1CCCCC1] | 16 | Piperidino Cis- Lactone | Phenyl | Phenyl |
| 161 | 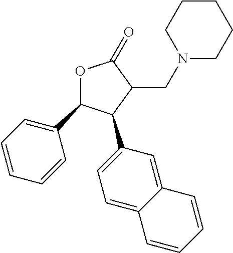 [SMILES: O=C1O[C@H](c2ccccc2)[C@H](c2ccc3ccccc3c2)C1CN1CCCCC1] | 16 | Piperidino Cis- Lactone | 2-naphthyl | Phenyl |
| 162 | 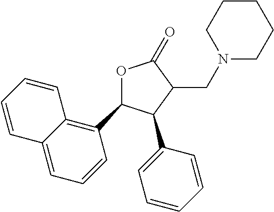 [SMILES: O=C1O[C@H](c2cccc3ccccc23)[C@H](c2ccccc2)C1CN1CCCCC1] | 8 | Piperidino Cis- Lactone | Phenyl | 1-naphthyl |
| 163 | 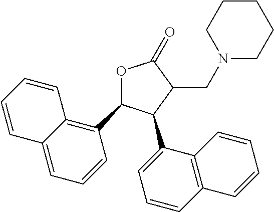 [SMILES: O=C1O[C@H](c2cccc3ccccc23)[C@H](c2cccc3ccccc23)C1CN1CCCCC1] | >64 | Piperidino Cis- Lactone | 1-naphthyl | 1-naphthyl |
| 164 | 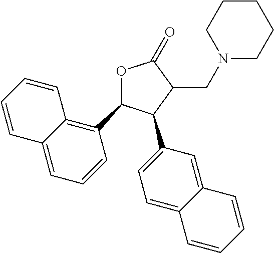 [SMILES: O=C1O[C@H](c2cccc3ccccc23)[C@H](c2ccc3ccccc3c2)C1CN1CCCCC1] | >64 | Piperidino Cis- Lactone | 2-naphthyl | 1-naphthyl |
| 167 | 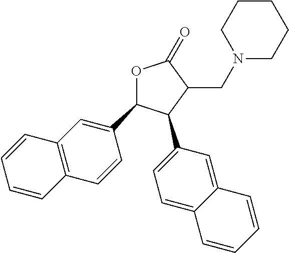 [SMILES: O=C1O[C@H](c2ccc3ccccc3c2)[C@H](c2ccc3ccccc3c2)C1CN1CCCCC1] | 2 | Piperidino Cis- Lactone | 2-naphthyl | 2-naphthyl |
| 169 | 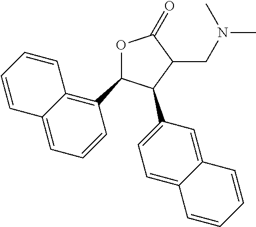 [SMILES: CN(C)CC1C(=O)O[C@H](c2cccc3ccccc23)[C@@H]1c1ccc2ccccc2c1] | 4 | Dimethylamino Cis-Lactone | 2-naphthyl | 1-naphthyl |
| 173 | 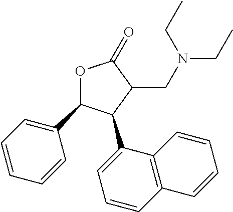 [SMILES: CCN(CC)CC1C(=O)O[C@H](c2ccccc2)[C@@H]1c1cccc2ccccc12] | 8 | Diethylamino Cis-Lactone | 1-naphthyl | Phenyl |
| 181 | 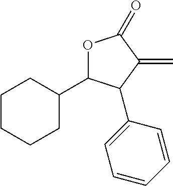 [SMILES: C=C1C(=O)OC(C2CCCCC2)C1c1ccccc1] | >64 | Methylene Cis- Lactone | phenyl | cyclohexyl |
| 182 | 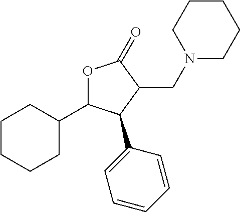 [SMILES: O=C1OC(C2CCCCC2)[C@H](c2ccccc2)C1CN1CCCCC1] | 32 | Piperidino Cis- Lactone | phenyl | cyclohexyl |
| 185 | 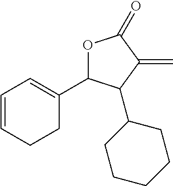 [SMILES: C=C1C(=O)OC(C2=CC=CCC2)C1C1CCCCC1] | >64 | Methylene Cis- Lactone | cyclohexyl | Phenyl |
| 186 | 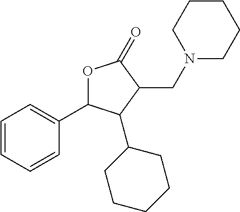 [SMILES: O=C1OC(c2ccccc2)C(C2CCCCC2)C1CN1CCCCC1] | >64 | Piperidino Cis- Lactone | cyclohexyl | Phenyl |
| 187 | 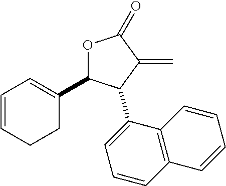 [SMILES: C=C1C(=O)O[C@H](C2=CC=CCC2)[C@H]1c1cccc2ccccc12] | 1 | Methylene Trans-Lactone | 1-naphthyl | Phenyl |

Summary Results: Compounds 144, 148, 149, 151, 155, 158, and 187 exhibited the most potent activity inhibiting the tested staphylococcal strain at an MIC value as low as 1-2 μg/mL.

The Minimum Inhibitory Concentration (MIC in μg/mL) of the Active Compounds (144, 148, 149, 151, 155, 158, and 187) Against a Panel of Clinically Important Gram-Positive Bacterial Pathogens.

Next, the spectrum of antibacterial activity of compounds 144, 148, 149, 151, 155, 158, and 187 was examined against a panel of clinically relevant Gram-positive bacterial pathogens including methicillin-resistant Staphylococcus aureus (MRSA), vancomycin-resistant Staphylococcus aureus (VRSA), vancomycin-resistant Enterococcus faecalis and Enterococcus faecium (VRE), and Streptococcus pneumoniae. Bacterial strains were grown aerobically overnight on tryptone soya agar plates at 37° C. Afterwards, a bacterial solution equivalent to 0.5 McFarland standard was prepared and diluted in cation-adjusted Mueller-Hinton broth (CAMHB) (for staphylococci). Enterococci and Streptococci were diluted in tryptone soya broth (TSB) to achieve a bacterial concentration of about 5×10⁵ CFU/mL and seeded in 96-well plates. Compounds and control drugs were added in the first row of 96-well plates and serially diluted with media containing bacteria. Plates were then, incubated aerobically at 37° C. for 18-20 hours (except for S. pneumoniae, which was incubated at 37° C. in presence of 5% CO₂ for 18-20 hours). MICs reported in Table 2 are the minimum concentration of the compounds and control drugs that completely inhibited the visual growth of bacteria.

TABLE 2
The minimum inhibitory concentration (MIC in μg/mL) of the
active compounds (144, 148, 149, 151, 155, 158, and 187)
and control drugs against a panel of clinically
important Gram-positive bacterial pathogens

| | Compounds/Control antibiotics | | | | | | | | |
|---|---|---|---|---|---|---|---|---|---|
| Bacterial strains | 144 | 148 | 149 | 151 | 155 | 158 | 187 | Van | Lin |
| S. aureus ATCC 6538 | 1 | 1 | 1 | 2 | 1 | 1 | 1 | 1 | 2 |
| S. aureus NRS 107 | 1 | 1 | 1 | 2 | 1 | 1 | 1 | 2 | 1 |
| MRSA NRS 385 | 1 | 1 | 1 | 1 | 1 | 1 | 1 | 1 | 2 |
| MRSA NRS 386 | 1 | 1 | 1 | 1 | 1 | 1 | 1 | 1 | 2 |
| MRSA NRS 123 | 1 | 1 | 1 | 2 | 1 | 1 | 2 | 1 | 2 |
| MRSA NRS 119 | 1 | 1 | 1 | 2 | 1 | 1 | 2 | 1 | >16 |
| VRSA 10 | 1 | 2 | 8 | 2 | 1 | 1 | 1 | >64 | 1 |
| VRSA 12 | 1 | 2 | 16 | 2 | 2 | 2 | 2 | >64 | 1 |
| Methicillin-resistant | 1 | 1 | 2 | 8 | 1 | 0.5 | 0.5 | 2 | 1 |
| S. epidermidis | | | | | | | | | |
| NRS101 | | | | | | | | | |
| Methicillin-resistant | 1 | 1 | 4 | 4 | 1 | 2 | 1 | 2 | 2 |
| S. pneumoniae | | | | | | | | | |
| ATCC 700677 | | | | | | | | | |
| S. pneumoniae | 1 | 1 | 4 | 4 | 1 | 2 | 1 | 2 | 2 |
| ATCC 51916 | | | | | | | | | |
| Enterococcus | >64 | >64 | >64 | >64 | >64 | >64 | >64 | 32 | 2 |
| faecalis ATCC | | | | | | | | | |
| 51299 (VRE)*¹ | | | | | | | | | |
| Enterococcus | 64 | >64 | >64 | >64 | >64 | >64 | >64 | >64 | 1 |
| faecium | | | | | | | | | |
| ATCC 700221 (VRE) | | | | | | | | | |

*¹VRE: vancomycin-resistant Enterococci; Lin, linezolid Van; vancomycin

Results: The compounds exhibited a potent antibacterial activity against the clinically important drug-resistant staphylococci and streptococci, inhibiting growth of the tested strains at concentrations of 0.5 to 2 μg/mL. They exhibited a potent activity against S. epidermidis, a common colonizer of the human skin, which represents the most common source of infections on implanted medical prosthetic devices. S. epidermidis infections are tough to be treated due to their huge ability to form strongly adherent biofilms that have intrinsic resistance to antibiotics and the host defense systems [26,27]. In addition, they exhibited a potent activity against S. pneumonia. Most importantly, the active compounds retained their antibacterial efficacy over vancomycin against vancomycin-resistant S. aureus (VRSA). The compounds did not exhibit any activity against vancomycin-resistant enterococci.

Time-Kill Assay of Compounds #148, #149, #151, #155, and #158 Against MRSA

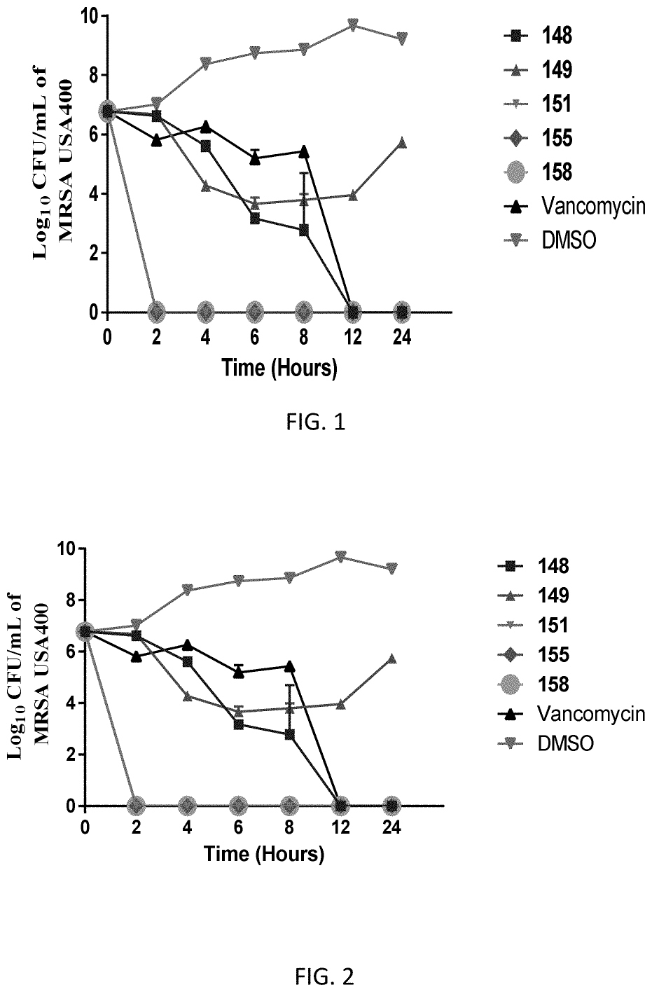

In order to the mode of killing of methylene lactone compounds, a time kill assay was performed against MRSA USA 400 as described previously [28,29]. MRSA USA400 cells in logarithmic growth phase (OD₆₀₀˜1.00) were diluted to 6.03×10⁶ colony-forming units (CFU/mL) and exposed to concentrations equivalent to 5×MIC (in triplicate) of 148, 149, 151, 155, 158 and vancomycin in Tryptic soy broth. Aliquots (100 μL) were collected from each treatment after 0, 2, 4, 6, 8, 12, and 24 hours of incubation at 37° C. and subsequently serially diluted in PBS. Bacteria were then transferred to Tryptic soy agar plates and incubated at 37° C. for 18-20 hours before viable CFU/mL was determined (FIG. 1).

FIG. 1: Killing kinetics of compounds 148, 149, 151, 155, and 158 (tested in triplicates at 5×MIC) against methicillin-resistant Staphylococcus aureus (MRSA USA 400) over a 24-hour incubation period at 37° C. DMSO (solvent for the compounds) served as a negative control and vancomycin served as a control drug. The error bars represent standard deviation values obtained from triplicate samples used for each compound/antibiotic studied.

Results: Compounds 148, 149, 151, 155, and 158 surpassed vancomycin; the drug of choice for the treatment of Staphylococcal infections in terms of the time required to exert their bactericidal activity. Vancomycin required 12 hours to exert its bactericidal activity resulting in complete eradication of the bacterial count. Advantageously, compounds 151, 155, and 158 exhibited a more rapid bactericidal activity than vancomycin, as they required 2 hours to bring about a complete eradication in the bacterial count. Compound 148 resulted in a 3-log₁₀-reduction (99.9%) in the bacterial count after 6 hours and required 12 hours to completely eradicate the bacteria. Compound 149 reduced the initial count by 3-log₁₀-reduction (99.9%) after 6 hours, but it was not able to completely eradicate it after 24 hours. This highlights the need of frequent dosing for this compound when used in clinical settings because the bacterial count showed only a slight reduction and started to increase after 12 hours.

Significance of Time-Kill Assay Results

The fact that compounds 151, 155, 158 completely kills the bacteria within 2 h is very significant. The rapid action within such a short time will prevent the bacteria from developing resistance. The slow action by the anti-bacterial allows the bacteria to understand the mechanism of action and overcome their bactericidal activity.

In Vitro Cytotoxicity Assessment Against HaCaT Cells

Compounds 144, 148, 149, 151, 155, and 158 were assayed (at concentrations of 16, 32, and 64 μg/mL) against a human keratinocytes cell line (HaCaT cells) to determine their in vitro potential toxic effect. Briefly, cells were cultured in Dulbecco's Modified Eagle Medium (DMEM) supplemented with 10% fetal bovine serum (FBS) and penicillin-streptomycin at 37° C. in presence of 5% CO₂. Compounds were added in the first row of the 96-well plate. Control cells received DMSO alone at a concentration equivalent to that in drug-treated wells. The cells were incubated with the compounds (in triplicate) in a 96-well plate at 37° C. in presence of 5% CO₂ for two hours. The assay reagent MTS 3-(4,5-dimethylthiazol-2-yl)-5-(3-carboxymethoxyphenyl)-2-(4-sulfophenyl)-2H-tetrazolium) (Promega, Madison, Wis., USA) was subsequently added and the plate was incubated for three to four hours. Absorbance readings (at OD₄₉₀) were recorded using a kinetic microplate reader (Molecular Devices, Sunnyvale, Calif., USA). The quantity of viable cells after treatment with each compound was expressed as a percentage of the viability to DMSO-treated control cells (average of triplicate wells±standard deviation).

FIG. 2. Analyzing the toxicity of compounds 144, 148, 149, 151, 155 and 158 (tested in triplicates at 16, 32 and 64 μg/mL) against human keratinocytes cells (HaCaT) using the MTS 3-(4,5-dimethylthiazol-2-yl)-5-(3-carboxymethoxyphenyl)-2-(4-sulfophenyl)-2H-tetrazolium) assay. Results are presented as percent viable cells relative to DMSO (negative control to determine a baseline measurement for the cytotoxic impact of each compound). The absorbance values represent an average of three samples analyzed for each compound. Error bars represent standard deviation values. The data were analyzed via a two-way ANOVA with post-hoc Dunnett's test for multiple comparisons. An asterisk (*) denotes statistical significance (P<0.05) between results for compounds and DMSO.

Results: Compounds 148, 155 and 158 were highly tolerable to HaCat cells at concentrations higher than 64 μg/mL. Compounds 144, 151 and 187 were tolerable to HaCat cells at concentrations as high as 32 μg/mL. This concentration represents 16 to 32 times their corresponding MICs against MRSA.

In Vitro Cytotoxicity Assessment Against Caco-2 Cells

Compounds 144, 148, 151, 155, 158, and 187 were assayed (at concentrations of 16, 32, and 64 μg/mL) against a human colorectal adenocarcinoma (Caco-2) cell to determine the potential toxic effect to mammalian cells in vitro. Briefly, cells were cultured in Dulbecco's Modified Eagle Medium (DMEM) supplemented with 10% fetal bovine serum (FBS), non-essential amino acids (1×), penicillin-streptomycin at 37° C. in presence of 5% CO₂. Control cells received DMSO alone at a concentration equal to that in drug-treated wells. The cells were incubated with the compounds (in triplicate) in a 96-well plate at 37° C. with 5% CO₂ for two hours. This was followed by the subsequent addition of the assay reagent MTS 3-(4,5-dimethylthiazol-2-yl)-5-(3-carboxymethoxyphenyl)-2-(4-sulfophenyl)-2H-tetrazolium) (Promega, Madison, Wis., USA) and the plate was incubated for three to four hours. Absorbance readings (at OD₄₉₀) were recorded using a kinetic microplate reader (Molecular Devices, Sunnyvale, Calif., USA). The quantity of viable cells after treatment with each compound was expressed as a percentage of the viability of DMSO-treated control cells (average of triplicate wells±standard deviation).

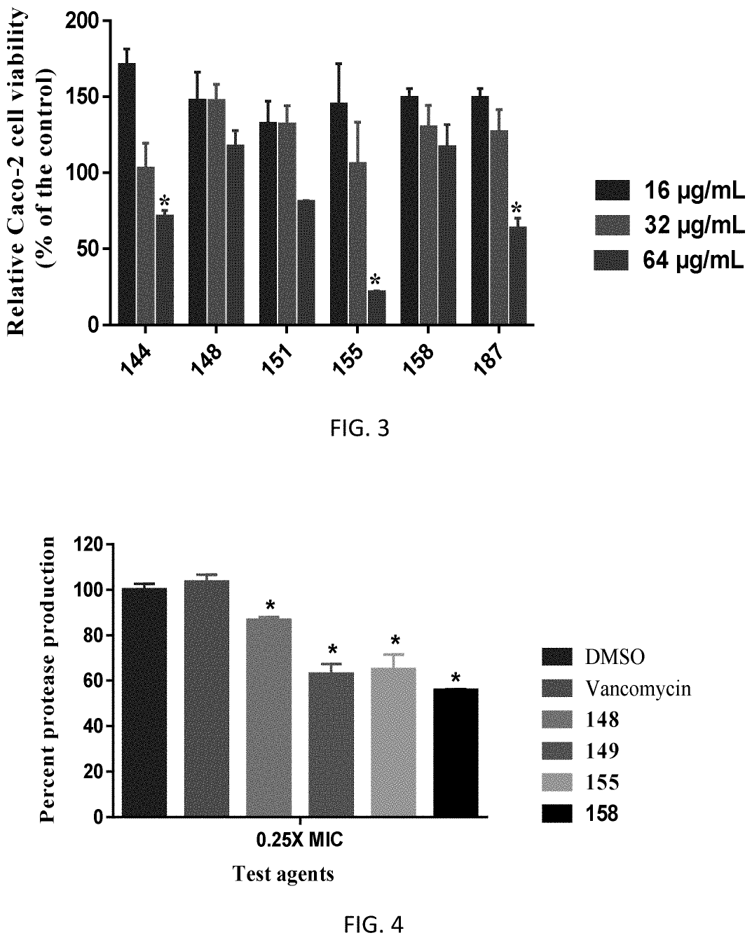

FIG. 3. Analyzing the toxicity of 144, 148, 151, 155, 158, and 187 compounds (tested in triplicates at 16, 32 and 64 μg/mL) against human colorectal cells (Caco-2) using the MTS 3-(4,5-dimethylthiazol-2-yl)-5-(3-carboxymethoxyphenyl)-2-(4-sulfophenyl)-2H-tetrazolium) assay. Results are presented as percent viable cells relative to DMSO (negative control) to determine a baseline measurement for the cytotoxic impact of each compound. The absorbance values represent an average of three samples analyzed for each compound. Error bars represent standard deviation values. The data were analyzed via a two-way ANOVA with post-hoc Dunnett's test for multiple comparisons. An asterisk (*) denotes statistical significance (P<0.05) between results for compounds and DMSO.

Results: Compounds 148, 151 and 158 were highly tolerable to Caco-2 cells at concentration higher than 64 μg/mL as represented by their 50% cytotoxic concentration (CC₅₀) where about 73%, 100%, 80%, 100%, and 65% of the cells were viable respectively. Thus, their 50% cytotoxic concentration (CC₅₀) is higher than 64 μg/mL. Compounds 155 was non-toxic to Caco-2 cells at a concentration as high as 32 μg/mL where 100% of the cells were viable at this concentration.

Inhibition of Staphylococcal Total Proteases Production

Staphylococcus aureus is a highly virulent and successful pathogen that causes a diverse array of diseases. Several community-associated MRSA (CA-MRSA) have appeared in the last decades. MRSA USA 300 had appeared in the last decade as one of the community-associated MRSA (CA-MRSA) strains and it is now representing the major clone in the USA [32, 33]. The reason behind this surprising success and diversity of staphylococci_in general_and MRSA USA300 specifically, as the primary CA-MRSA may be attributed to the differential expression of genomic elements including the hemolysins, enterotoxins, and extracellular proteases [34-36]. S. aureus possesses 10 major different secreted proteolytic enzymes representing different protease classes including metalloproteases, serine proteases, cysteine proteases and six serine-like proteases [37,38]. Proteases are considered as one of the most important virulence factors of bacteria in general and specifically S. aureus. They have been shown to cleave specific host proteins such as human fibronectin, fibrinogen, and kininogen contributing to the ability of S. aureus to disseminate [38]. Furthermore, they destruct immunoglobulins which protect mucous membranes as well as disrupt tight junction between host epithelial cells leading to tissue invasion and damage [39-41]. Moreover, it has been demonstrated that secreted proteases modulate the stability of the bacterial virulence determinants. They can cleave secreted toxins in order to regulate the abundance of virulence factors depending on the specific niche encountered within the host [42,43]. In this context, we examined the effect of our compounds in inhibiting the total proteases production in MRSA USA300 which is well-known to hyperproduce secreted proteases.

The modified skimmed milk method was utilized to investigate the protease inhibitory activities of BHS compounds as described previously [44]. In brief, overnight culture of MRSA300 in TSB, with and without subinhibitory concentrations of compounds (in triplicate), was centrifuged at 14000×g for 10 minutes. Then, a total volume of 500 μL of culture supernatant was incubated with 1 mL skimmed milk (1.25%) at 37° C. for 1 hour. Afterwards, the OD₆₀₀ was measured to indicate the degree of clearance of skimmed milk. Higher OD values indicate lower proteolytic activity and greater protease inhibitory activity.

FIG. 4. Influence of BHS compounds on the staphylococcal total protease production. OD₆₀₀ was measured following overnight culturing of MRSA USA300 in TSB in presence and absence of 0.25×MIC of 148, 149, 155, 158 and vancomycin followed by incubation of supernatants with skim milk for 1 hour at 37° C. The Data are presented as percent production (means±standard deviation). DMSO (the solvent for the compounds that served as a negative control). Vancomycin served as the control antibiotic. The values represent an average (means±standard deviation) of four samples analyzed for each compound/antibiotic. Error bars represent standard deviation values. An asterisk (*) denotes statistical significance (P<0.05) between results for compounds 148, 149, 155, 158 and vancomycin analyzed via unpaired t test (P<0.5).

Results: Total protease production was decreased from 100% in untreated isolate to 87%, 63%, 65% and 56% in 148, 149, 155 and 158-treated bacteria respectively. On the other hand, vancomycin was ineffective in inhibiting MRSA USA300 protease production at 0.25×MIC.

In Vitro Cytotoxicity Assessment Against J774 Cells

Compounds were assayed [30] against a murine macrophage (J774) cell line to determine their in vitro potential toxic effect. Cells were cultured in Dulbecco's Modified Eagle Medium (DMEM) supplemented with 10% FBS at 37° C. with 5% CO₂. Control cells received DMSO alone at a concentration equal to that in drug-treated cell samples. The cells were incubated with the compounds (in triplicate) in a 96-well plate at 37° C. with 5% CO₂ for 24 hours. The assay reagent MTS; 3-(4,5-dimethylthiazol-2-yl)-5-(3-carboxymethoxyphenyl)-2-(4-sulfophenyl)-2H-tetrazolium) (Promega, Madison, Wis., USA) was subsequently added and the plate was incubated for three to four hours. Absorbance readings (at OD₄₉₀) were recorded using a kinetic microplate reader (Molecular Devices, Sunnyvale, Calif., USA). The quantity of viable cells after treatment with each compound was expressed as a percentage of the viability relative to DMSO-treated control cells (average of triplicate wells±standard deviation).

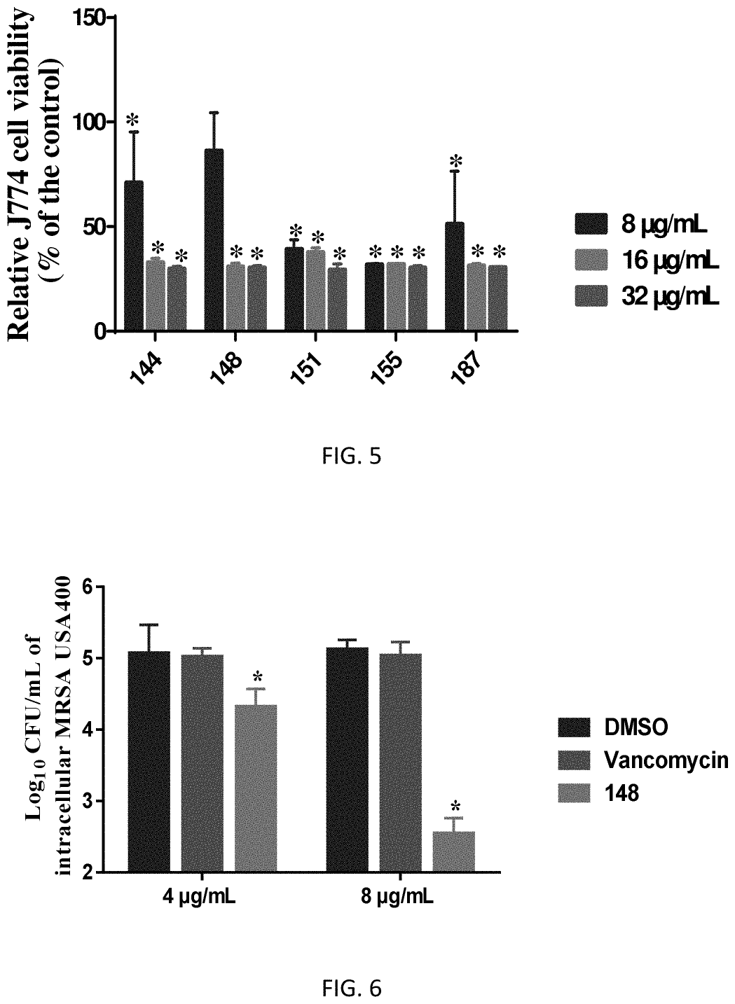

FIG. 5. Toxicity analysis of compounds 144, 148, 151, 155 and 187 against murine macrophage (J774) cells using the MTS 3-(4,5-dimethylthiazol-2-yl)-5-(3-carboxymethoxyphenyl)-2-(4-sulfophenyl)-2H-tetrazolium) assay. Results are presented as percent viable mammalian cells (measured as average absorbance ratio relative to DMSO). The absorbance values represent an average of a minimum of three samples analyzed for each compound. Error bars represent standard deviation values for the absorbance values.

Results: Compounds 148 was tolerable to J774 up until a concentration of 8 μg/mL. Thus, we chose concentrations ≤8 μg/mL for testing their intracellular clearance activity.

Intracellular Infection of J774 Cells with MRSA and Treatment with 148 and 149 Compounds

The ability of the compound 148 to reduce the burden of intracellular MRSA was evaluated utilizing previously described methods [30,31]. Murine macrophage cells (J774) were cultured in Dulbecco's Modified Eagle Medium (DMEM) supplemented with 10% FBS at 37° C. with CO₂ (5%). J774 cells were exposed to MRSA USA400 cells at a multiplicity of infection of approximately 10:1. After 1 hour of infection, J774 cells were washed with gentamicin (200 μg/mL) to kill extracellular MRSA. The compounds or vancomycin (at 4×MIC and 8×MIC) were subsequently added to each well (four replicates per test agent). Control cells received DMSO at a concentration equal to that in drug-treated cell samples. After 24 hours-incubation at 37° C. with 5% CO₂, the test agents were removed. J774 cells were washed and subsequently lysed using 0.1% Triton-X. The solution was serially diluted in phosphate-buffered saline and transferred to TSA plates in order to determine viable MRSA CFU inside the J774 cells. Plates were incubated at 37° C. for 18-22 hour before counting viable CFU/mL. Statistical significance was assessed with two-way ANOVA, with post hoc Dunnet's multiple comparisons test (P<0.05), utilizing GraphPad Prism 6.0 (GraphPad Software, La Jolla, Calif.).

FIG. 6. Examination of the activity of compounds 148 on the clearance of intracellular MRSA present inside infected murine macrophage (J774) cells. Data has been presented as log₁₀ colony forming units of MRSA USA400 per mL inside infected murine macrophages after treatment with 4×MIC and 8×MIC of either compounds 148 or vancomycin (tested in triplicates) for 24 hours. Data were analyzed via two-way ANOVA, with post hoc Dunnet's multiple comparisons test (P<0.05), utilizing GraphPad Prism 6.0 (GraphPad Software, La Jolla, Calif.). Asterisks (*) represent significant difference between treatment of J774 cells with compound 148 in comparison to vancomycin.

Results: Given its high molecular weight and complex structure, vancomycin is unable to sufficiently accumulate inside macrophage cells and clear the intracellular MRSA infection. At 4 μg/mL and 8 μg/mL, vancomycin, as expected, was not able to reduce the presence of MRSA inside infected J774 cells, even after 24 hours of treatment. As depicted in FIG. 6, at 4 μg/mL, 148 generated a 0.8-log₁₀ reduction (equivalent to 84.9% reduction) of intracellular MRSA. This increased to reach about 2.52 log₁₀ reduction (equivalent to 99.7% reduction) of intracellular MRSA when its concentration was increased by one fold (8 μg/mL). The results collectively indicated that methylene lactone 148 had the ability to gain entry into macrophage cells at low concentrations (4 and 8 μg/mL) and this concentration is capable of significantly reducing the burden of MRSA inside them.

Those skilled in the art will recognize that numerous modifications can be made to the specific implementations described above. The implementations should not be limited to the particular limitations described. Other implementations may be possible.

It is intended that that the scope of the present methods and compositions be defined by the following claims. However, it must be understood that this disclosure may be practiced otherwise than is specifically explained and illustrated without departing from its spirit or scope. It should be understood by those skilled in the art that various alternatives to the embodiments described herein may be employed in practicing the claims without departing from the spirit and scope as defined in the following claims.

### REFERENCES

1. CDC, Antibiotic Resistance Threats in the United States, 2013. Centers for Disease Control and Prevention. p. 1-114. 2013.
2. CDC, Antibiotic Resistance Threats in the United States, 2015. Centers for Disease Control and Prevention. Updated report. 2015.
3. WHO Review: Antimicrobial resistance—Global strategic direction. 2017.
4. Abb, J., In vitro activities of tigecycline, daptomycin, linezolid and quinupristin/dalfopristin against glycopeptide-resistant Enterococcus faecium. International Journal of Antimicrobial Agents, 2007. 29(3): p. 358-360.
5. Manzella, J. P., Quinupristin-dalfopristin: A new antibiotic for severe gram-positive infections. American Family Physician, 2001. 64(11): p. 1863-1866.
6. Editorial, Wanted: a reward for antibiotic development. Nat Biotechnol, 2018. 36(7): p. 555.
7. Hoffmann, H. M. R. and J. Rabe, Synthesis and biological-activity of alpha-methylene-gamma-butyrolactones. Angewandte Chemie-International Edition in English, 1985. 24(2): p. 94-110.
8. Kagan, H. B., et al., Structure of psilostachyin c a new sesquiterpene dilactone from ambrosia psilostachya dc. Journal of Organic Chemistry, 1966. 31(5): p. 1629-&.
9. Wang, L. T., et al., New cytotoxic cembranolides from the soft coral Lobophytum michaelae. Chemical & Pharmaceutical Bulletin, 2007. 55(5): p. 766-770.
10. Wang, Y., et al., Ainsliatrimers A and B, the First Two Guaianolide Trimers from Ainsliaea fulvioides. Organic Letters, 2008. 10(24): p. 5517-5520.
11. Yamada, M., et al., Germacranolides from Calea urticifolia. Phytochemistry, 2004. 65(23):
p. 3107-3111.
12. Kupchan, S. M., et al., Reactions of alpha methylene lactone tumor inhibitors with model biological nucleophiles. Science, 1970. 168(3929): p. 376-&.
13. Kitson, R. R. A., A. Millemaggi, and R. J. K. Taylor, The Renaissance of alpha-Methylene-gamma-butyrolactones: New Synthetic Approaches. Angewandte Chemie-International Edition, 2009. 48(50): p. 9426-9451.
14. Cavallito, C. J., D. M. Fruehauf, and J. H. Bailey, Lactone aliphatic acids as antibacterial agents. Journal of the American Chemical Society, 1948. 70(11): p. 3724-3726.
15. Blakeman, J. P. and P. Atkinson, Anti-microbial properties and possible role in host-pathogen interactions of parthenolide, a sesquiterpene lactone isolated from glands of chrysanthemum-parthenium. Physiological Plant Pathology, 1979. 15(2): p. 183-+.
16. Picman, A. K., Biological-activities of sesquiterpene lactones. Biochemical Systematics and Ecology, 1986. 14(3): p. 255-281.
17. Park, B. K., et al., Methylenolactocin, a novel antitumor antibiotic from penicillium sp. Journal of Antibiotics, 1988. 41(6): p. 751-758.
18. Goren, N., J. Jakupovic, and S. Topal, Sesquiterpene lactones with antibacterial activity from tanacetum-argyrophyllum var argyrophyllum. Phytochemistry, 1990. 29(5): p. 1467-1469.
19. Saroglou, V., et al., Sesquiterpene Lactones from Anthemis melanolepis and Their Antibacterial and Cytotoxic Activities. Prediction of Their Pharmacokinetic Profile. Journal of Natural Products, 2010. 73(2): p. 242-246.
20. Ramachandran, P. V., et al., Tailored alpha-methylene-gamma-butyrolactones and their effects on growth suppression in pancreatic carcinoma cells. Bioorganic & Medicinal Chemistry Letters, 2010. 20(22): p. 6620-6623.
21. Hejchman, E., R. D. Haugwitz, and M. Cushman, Synthesis and cytotoxicity of water-soluble ambrosin prodrug candidates. Journal of Medicinal Chemistry, 1995. 38(17): p. 3407-3410.
22. Ramachandran, P. V., et al., Synthetic alpha-(aminomethyl)-gamma-butyrolactones and their anti-pancreatic cancer activities. Bioorganic & Medicinal Chemistry Letters, 2013. 23(24): p. 6911-6914.
23. Ramachandran, P. V. and D. R. Nicponski, Diastereoselective synthesis of alpha-(aminomethyl)-gamma-butyrolactones via a catalyst free aminolactonization. Chemical Communications, 2014. 50(96): p. 15216-15219.
24. Nasim, S. and P. A. Crooks, Antileukemic activity of aminoparthenolide analogs. Bioorg Med Chem Lett, 2008. 18(14): p. 3870-3.
25. CLSI, Methods for Dilution Antimicrobial Susceptibility Tests for Bacteria That Grow Aerobically; Approved Standard. Vol. Ninth Edition M07-A9. 32 No. 2. January 2012.
26. Costerton, J. W., P. S. Stewart, and E. P. Greenberg, Bacterial biofilms: a common cause of persistent infections. science, 1999. 284(5418): p. 1318-1322.
27. Otto, M., Staphylococcus epidermidis-the'accidental'pathogen. Nature reviews microbiology, 2009. 7(8): p. 555.
28. Hagras, M., et al., Biphenylthiazole antibiotics with an oxadiazole linker: An approach to improve physicochemical properties and oral bioavailability. Eur J Med Chem, 2018. 143: p. 1448-1456.
29. Mohammad, H., et al., Repurposing niclosamide for intestinal decolonization of vancomycin-resistant enterococci. International Journal of Antimicrobial Agents, 2018. 51(6): p. 897-904.
30. Hagras, M., et al., Naphthylthiazoles: Targeting Multidrug-Resistant and Intracellular Staphylococcus aureus with Biofilm Disruption Activity. ACS infectious diseases, 2018. 4(12): p. 1679-1691.
31. Pei, Y. H., et al., Particle engineering for intracellular delivery of vancomycin to methicillin-resistant Staphylococcus aureus (MRSA)-infected macrophages. Journal of Controlled Release, 2017. 267: p. 133-143.
32. Limbago, B., et al., Characterization of Methicillin-Resistant Staphylococcus aureus Isolates Collected in 2005 and 2006 from Patients with Invasive Disease: a Population-Based Analysis. Journal of Clinical Microbiology, 2009. 47(5): p. 1344-1351.
33. Tenover, F. C., et al., Characterization of Staphylococcus aureus isolates from nasal cultures collected from individuals in the United States in 2001 to 2004. Journal of Clinical Microbiology, 2008. 46(9): p. 2837-2841.
34. Kobayashi, S. D. and F. R. Deleo, An update on community-associated MRSA virulence. Current Opinion in Pharmacology, 2009. 9(5): p. 545-551.
35. Li, M., et al., Evolution of virulence in epidemic community-associated methicillin-resistant Staphylococcus aureus. Proceedings of the National Academy of Sciences of the United States of America, 2009. 106(14): p. 5883-5888.
36. Montgomery, C. P., S. Boyle-Vavra, and P. V. Adem, Comparison of virulence in community-associated methicillin-resistant Staphylococcus aureus pulsotypes USA300 and USA400 in a rat model of pneumonia (vol 198, pg 561, 2008). Journal of Infectious Diseases, 2008. 198(11): p. 1725-1725.
37. Reed, S. B., et al., Molecular characterization of a novel Staphylococcus aureus serine protease operon. Infection and Immunity, 2001. 69(3): p. 1521-1527.
38. Shaw, L., et al., The role and regulation of the extracellular proteases of Staphylococcus aureus. Microbiology-Sgm, 2004. 150: p. 217-228.
39. Heras, B., M. J. Scanlon, and J. L. Martin, Targeting virulence not viability in the search for future antibacterials. British journal of clinical pharmacology, 2015. 79(2): p. 208-215.
40. Prokesova, L., et al., Cleavage of Human-Immunoglobulins by Serine Proteinase from Staphylococcus-Aureus. Immunology Letters, 1992. 31(3): p. 259-265.
41. Massimi, I., et al., Identification of a novel maturation mechanism and restricted substrate specificity for the SspB cysteine protease of Staphylococcus aureus. Journal of Biological Chemistry, 2002. 277(44): p. 41770-41777.
42. Lindsay, J. A. and S. J. Foster, Interactive regulatory pathways control virulence determinant production and stability in response to environmental conditions in Staphylococcus aureus. Molecular and General Genetics, 1999. 262(2): p. 323-331.
43. Gonzalez, D. J., et al., Novel Phenol-soluble Modulin Derivatives in Community-associated Methicillin-resistant Staphylococcus aureus Identified through Imaging Mass Spectrometry. Journal of Biological Chemistry, 2012. 287(17): p. 13889-13898.
44. Skindersoe, M. E., et al., Effects of antibiotics on quorum sensing in Pseudomonas aeruginosa. Antimicrob Agents Chemother, 2008. 52(10): p. 3648-63.
45. Kim, K. H.; Lee, H. S.; Kim, S. H.; Lee, K. Y.; Lee, J-E; Kim, J. N. Expedient One-Pot Synthesis of γ-Hydroxybutenolides Starting from Baylis-Hillman Adducts: Lactonization, Isomerization, and Aerobic Oxidation of α-Methylene-γ-hydroxyester; Bull. Korean. Chem. Soc. 2009, 30, 1012.
46. Ishiyama, T.; Ahiko, T-A.; Miyaura, N. Acceleration Effect of Lewis Acid in Allylboration of Aldehydes: Catalytic, Regiospecific, Diastereospecific, and Enantioselective Synthesis of Homoallyl Alcohols. J. Am. Chem. Soc. 2002, 124, 12414.
47. Ramachandran, P. V.; Garner, G.; Pratihar, D. Synthesis of (E)- and (Z)-α-Alkylidene-γ-aryl-γ-butyrolactones via Alkenylalumination of Oxiranes Org. Lett. 2007, 9, 4753.
48. Park, B. R.; Kim, K. H.; Kim, J. N. An efficient synthesis of α-methylene-γ-butyrolactones from Baylis-Hillman adducts via an In-mediated Barbier reaction and stereoselective lactonization under MeSO2 Cl/Et3N conditions. Tetrahedron Lett. 2010, 51, 6568.
49. Kennedy, J. W. J.; Hall, D. G. Dramatic Rate Enhancement with Preservation of Stereo specificity in the First Metal-Catalyzed Additions of Allylboronates. J. Am. Chem. Soc. 2002, 124, 11586.

1. A compound for treating a patient with an infection having a formula (I): 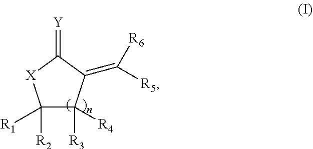
or a pharmaceutically acceptable salt thereof, wherein
X═O, NH, NR, S; Y═O, NH, NR, S, wherein R is a C1-C6 alkyl; n=1, 2, 3, 4; and
R₁-R₆ are a substituent independently selected from the group consisting of hydrogen, halogen, hydroxyl, an alkyl, alkenyl, alkynyl, heteroalkyl, heteroalkenyl, heteroalkynyl, heterocyclyl, cycloalkyl, cyclo alkenyl, cycloheteroalkyl, cycloheteroalkenyl, acyl, aryl, heteroaryl, arylalkyl, arylalkenyl, or arylalkynyl, each of which is optionally substituted.

2. The compound according to claim 1, wherein the compound has a formula (II): 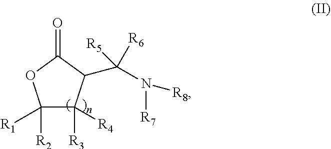
wherein
n=1, 2, 3, 4; and
R₁-R₆ are a substituent independently selected from the group consisting of hydrogen, halogen, hydroxyl, an alkyl, alkenyl, alkynyl, heteroalkyl, heteroalkenyl, heteroalkynyl, heterocyclyl, cycloalkyl, cyclo alkenyl, cycloheteroalkyl, cycloheteroalkenyl, acyl, aryl, heteroaryl, arylalkyl, arylalkenyl, or arylalkynyl, each of which is optionally substituted.

3. The compound according to claim 2, wherein R₅ and R₆ are hydrogen.

4. The compound according to claim 2, wherein said compounds are compounds #1-4, 10, 12-13, 19-25, 36-40, 42-44, 49-56, 59, 61, 64-75, 77-79, 83-90, 92-94, 97-100, 102-107, 109-125, 150-158, 181, 185, and 187 as shown in Table 1.

5. A compound for treating a patient with an infection having a formula (III): 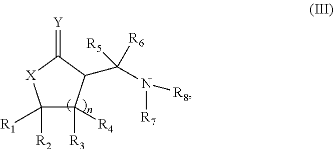
or a pharmaceutically acceptable salt thereof,
wherein
X═O, NH, NR, S; Y═O, NH, NR, S, wherein R is a C1-C6 alkyl; n=1, 2, 3, 4;
R₁-R₆ are a substituent independently selected from the group consisting of hydrogen, halogen, hydroxyl, an alkyl, alkenyl, alkynyl, heteroalkyl, heteroalkenyl, heteroalkynyl, heterocyclyl, cycloalkyl, cyclo alkenyl, cycloheteroalkyl, cycloheteroalkenyl, acyl, aryl, heteroaryl, arylalkyl, arylalkenyl, or arylalkynyl, each of which is optionally substituted; and
R₇-R₈ are a substituent independently selected from the group consisting of hydrogen, an alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted; or R₇-R₈ are part of a ring system with or without one or more heteroatoms.

6. The compound according to claim 4, wherein the compound has a formula (IV): 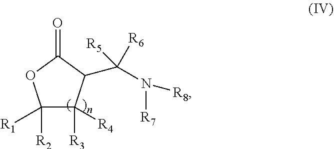
wherein
n=1, 2, 3, 4;
R₁-R₆ are a substituent independently selected from the group consisting of hydrogen, halogen, hydroxyl, an alkyl, alkenyl, alkynyl, heteroalkyl, heteroalkenyl, heteroalkynyl, heterocyclyl, cycloalkyl, cyclo alkenyl, cycloheteroalkyl, cycloheteroalkenyl, acyl, aryl, heteroaryl, arylalkyl, arylalkenyl, or arylalkynyl, each of which is optionally substituted; and
R₇-R₈ are a substituent independently selected from the group consisting of hydrogen, an alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted; or R₇-R₈ are part of a ring system with or without one or more heteroatoms.

7. The compound according to claim 6, wherein R₅ and R₆ are hydrogen.

8. The compound according to claim 6, wherein said compounds are compound #5-9, 11, 14-18, 26-35, 41, 45-48, 57-58, 62-63, 76, 80, 82, 91, 95, 96, 101, 108, 130, 132, 137-140, 159, 161-164, 167, 169, 173, 182, and 186 as shown in Table 1.

9. A pharmaceutical composition comprising one or more compounds of claim 1, together with one or more pharmaceutically acceptable diluents, excipients or carriers.

10. A pharmaceutical composition comprising nanoparticles of one or more compounds of claim 1, together with one or more diluents, excipients or carriers.

11. A pharmaceutical composition comprising one or more compounds of claim 4, together with one or more pharmaceutically acceptable diluents, excipients or carriers.

12. A pharmaceutical composition comprising nanoparticles of one or more compounds of claim 4, together with one or more diluents, excipients or carriers.

13. A method for treating a patient with an infection comprising the step of administering a therapeutically effective amount of one or more compounds of formula (I) or (III), or a pharmaceutically acceptable salt thereof, together with one or more carriers, diluents, or excipients, to a patient in need of relief from said infection: 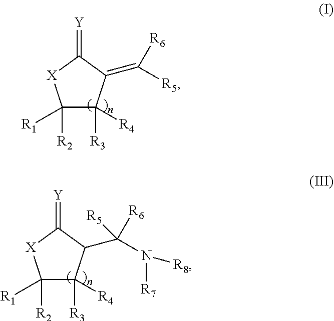
wherein
X═O, NH, NR, S; Y═O, NH, NR, S, wherein R is a C1-C6 alkyl; n=1, 2, 3, 4; and
R₁-R₆ are a substituent independently selected from the group consisting of hydrogen, halogen, hydroxyl, an alkyl, alkenyl, alkynyl, heteroalkyl, heteroalkenyl, heteroalkynyl, heterocyclyl, cycloalkyl, cyclo alkenyl, cycloheteroalkyl, cycloheteroalkenyl, acyl, aryl, heteroaryl, arylalkyl, arylalkenyl, or arylalkynyl, each of which is optionally substituted; and
R₇-R₈ are a substituent independently selected from the group consisting of hydrogen, an alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl.

14. The method according to claim 13, wherein the compound has a formula (II): 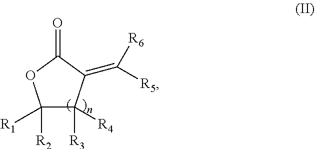
wherein
n=1, 2, 3, 4; and
R₁-R₆ are a substituent independently selected from the group consisting of hydrogen, halogen, hydroxyl, an alkyl, alkenyl, alkynyl, heteroalkyl, heteroalkenyl, heteroalkynyl, heterocyclyl, cycloalkyl, cyclo alkenyl, cycloheteroalkyl, cycloheteroalkenyl, acyl, aryl, heteroaryl, arylalkyl, arylalkenyl, or arylalkynyl, each of which is optionally substituted.

15. The method of claim 14, wherein said infection is a bacterial or a viral infection.

16. The method of claim 15, wherein said bacterial infection is caused by a Gram-positive bacteria.

17. The method according to claim 13, wherein the compound has a formula (IV): 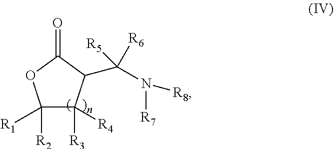
wherein
n=1, 2, 3, 4;
R₁-R₆ are a substituent independently selected from the group consisting of hydrogen, halogen, hydroxyl, an alkyl, alkenyl, alkynyl, heteroalkyl, heteroalkenyl, heteroalkynyl, heterocyclyl, cycloalkyl, cyclo alkenyl, cycloheteroalkyl, cycloheteroalkenyl, acyl, aryl, heteroaryl, arylalkyl, arylalkenyl, or arylalkynyl, each of which is optionally substituted; and
R₇-R₈ are a substituent independently selected from the group consisting of hydrogen, an alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, and heteroaryl.

18. The method of claim 17, wherein said infection is a bacterial or a viral infection.

19. The method of claim 18, wherein said Gram-positive bacteria comprises methicillin-resistant Staphylococcus aureus (MRSA), vancomycin-resistant Staphylococcus aureus (VRSA), vancomycin-resistant Enterococcus faecalis and Enterococcus faecium (VRE), and Streptococcus pneumoniae.

20. The method according claim 13, wherein said compounds are compound #1-125, 130, 132, 137-140, 150-164, 167, 169, 173, 181, 182, and 185-187.
